(19) United States
(12) Patent Application Publication
(10) Pub. No.: US 2023/0201236 A1
(43) Pub. Date: Jun. 29, 2023

(54) SPIROCYCLIC NUCLEOSIDE ANALOGUES FOR THE TREATMENT OF HEPATITIS E

(71) Applicant: JANSSEN PHARMACEUTICALS, INC., Titusville, NJ (US)

(72) Inventors: Tim Hugo Maria JONCKERS, Philadelphia, PA (US); Frederik PAUWELS, Waasmunster (BE); Yannick DEBING, Bilzen (BE)

(21) Appl. No.: 17/995,948

(22) PCT Filed: Apr. 13, 2021
(86) PCT No.: PCT/EP2021/059514

§ 371 Date: Oct. 11, 2022

(30) Foreign Application Priority Data
Apr. 14, 2020 (EP) 20169495.7

(51) Int. Cl. A61K 31/7072; A61P 31/14

(52) U.S. Cl. CPC A61K 31/7072; A61P 31/14

(57) ABSTRACT

The present disclosure is directed toward spirocyclic nucleoside analogs, compositions comprising these compounds, and their use for treating hepatitis E infections.

### BACKGROUND

Hepatitis E virus (HEV) is believed to be the cause of about 20 million human infections a year and the most common cause of acute hepatitis and jaundice worldwide. Immuno-compromised patients are a significant population at risk for chronic HEV infection. Acute HEV infections tend to be self-limiting, but HEV genotype 3 can persist in immune-compromised patients, especially organ transplant recipients, leading to chronic hepatitis, cirrhosis and/or liver failure.

HEV is a positive-sense, single-stranded, nonenveloped, RNA icosahedral virus classified in the genus Orthohepevirus and the family Hepeviridae. Although HEV genotype 1 and 2 infect only humans, genotypes 3 and 4 also infect swine and other types of animals. Each of the four genotypes is classified into multiple subtypes.

HEV infections have been treated by ribavirin (RBV) and pegylated interferon-α with varying success. Accordingly, there is still a need for safe, tolerable and effective treatment options for HEV infections.

### SUMMARY

Provided herein are methods of ameliorating and/or treating a Hepatitis E (HEV) infection, as well as compounds for use in such treatment.

In an aspect, provided herein are compounds for use in treating a hepatitis E infection in a subject in need thereof, wherein the compound is a compound of formula (I):

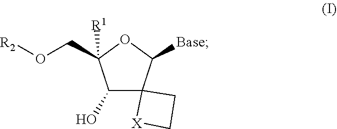

or a pharmaceutically acceptable salt thereof;

wherein:

Base is selected from the group consisting of (b-1), (b-2), (b-3), (b-4), (b-5), (b-6), (b-7), and (b-8):

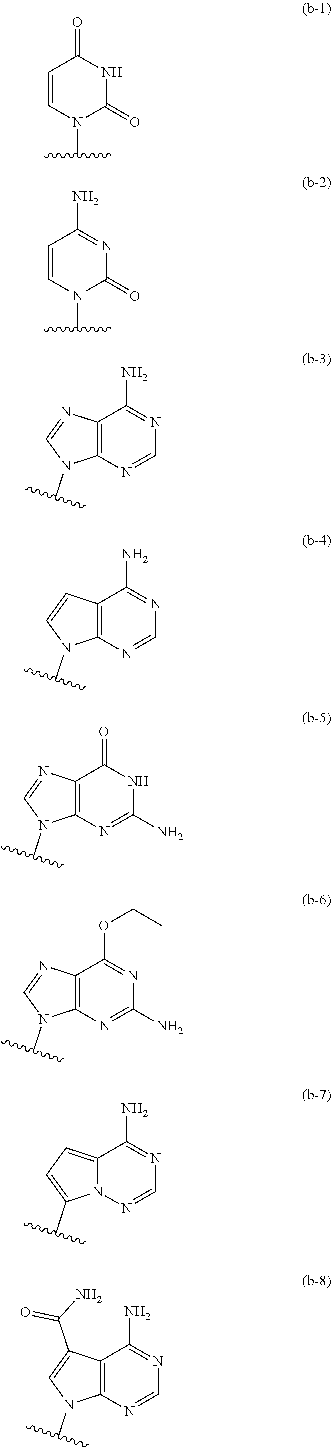

X is selected from the group consisting of O and S;

R¹ is selected from the group consisting of H, F, and N₃; and

R² is selected from the group consisting of (f-1) and (f-2):

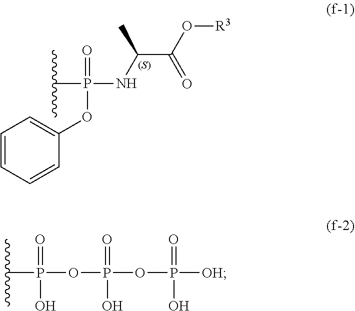

R³ is C₁₋₄alkyl.

In an embodiment, Base is (b-1). In another embodiment, R² is (f-1). In yet another embodiment, Base is (b-1), X is S, R² is (f-1) and R³ is isopropyl. In still another embodiment, Base is (b-1), X is O, R² is (f-1) and R³ is butyl.

In an embodiment, the compound is selected from the group consisting of:

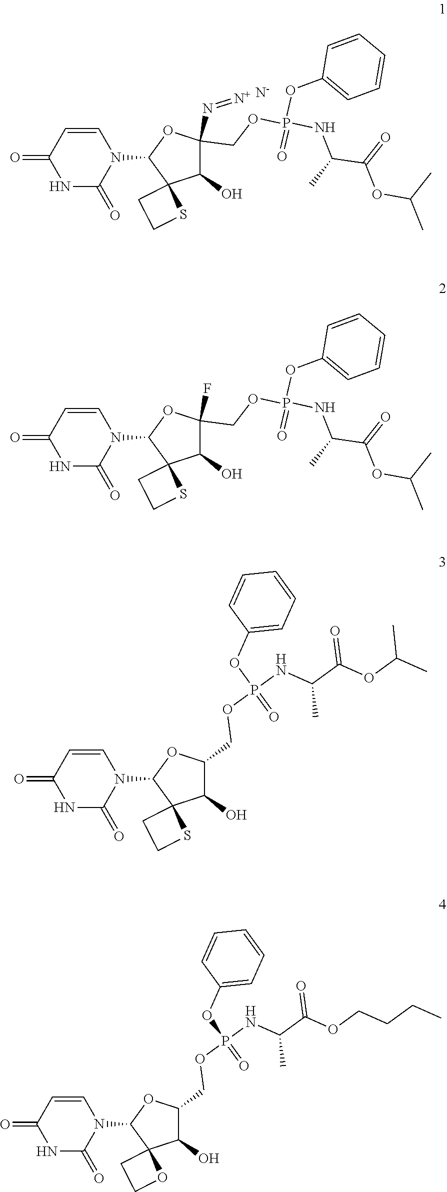

or a pharmaceutically acceptable salt thereof.

In another aspect, provided herein are pharmaceutical compositions for use in treating a hepatitis E infection in a subject in need thereof, which comprises a compound of Formula (I), and a pharmaceutically acceptable vehicle.

In an embodiment, the hepatitis E infection is a chronic HEV infection. In another embodiment, the HEV infection is of genotype 1, genotype 2 or genotype 3. In yet another embodiment, the subject is a pregnant woman, an immune-compromised subject or immune-deficient subject.

In yet another aspect, provided herein are methods of treating a hepatitis E infection in a subject in need thereof, the method comprising administering to the subject a therapeutically effective amount of a compound of formula (I):

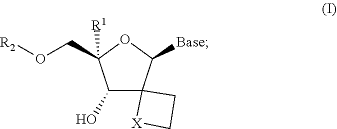

or a pharmaceutically acceptable salt thereof;

wherein:

Base is selected from the group consisting of (b-1), (b-2), (b-3), (b-4), (b-5), (b-6), (b-7), and (b-8):

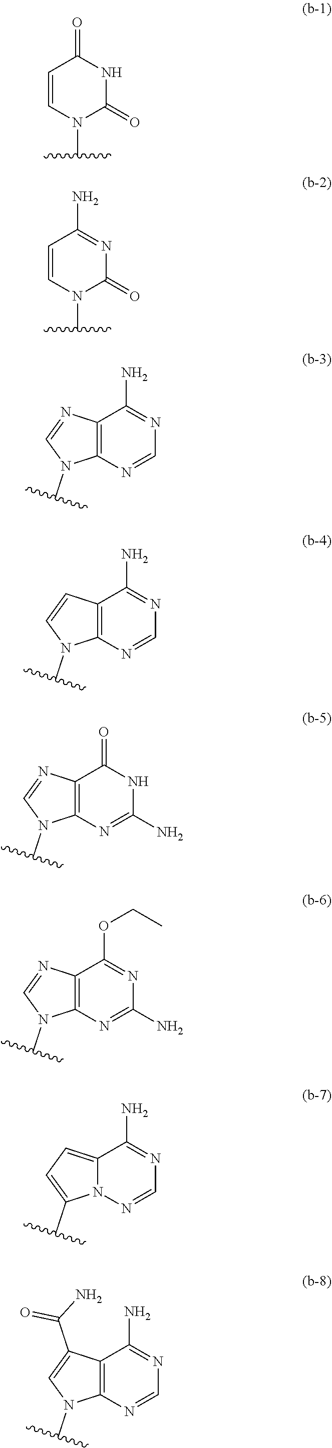

X is selected from the group consisting of O and S;

R¹ is selected from the group consisting of H, F, and N₃; and

R² is selected from the group consisting of (f-1) and (f-2):

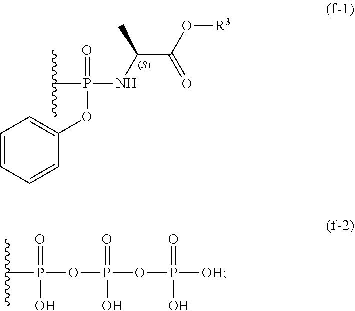

and

R³ is C₁₋₄alkyl.

In an embodiment, Base is (b-1). In another embodiment, R² is (f-1). In yet another embodiment, Base is (b-1), X is S, R² is (f-1) and R³ is isopropyl. In still another embodiment, Base is (b-1), X is O, R² is (f-1) and R³ is butyl.

In an embodiment, the compound is selected from the group consisting of:

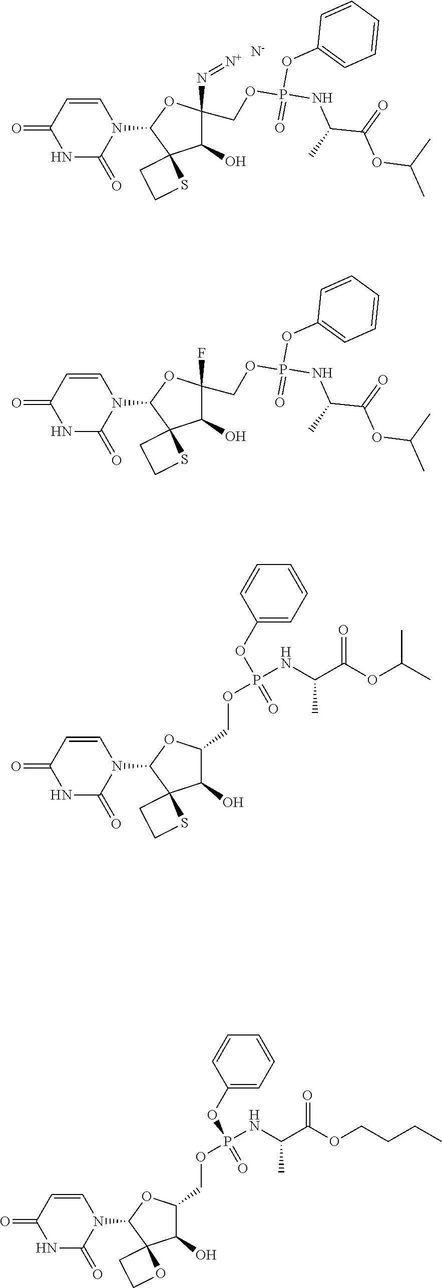

or a pharmaceutically acceptable salt thereof.

In an embodiment, the hepatitis E infection is a chronic HEV infection. In another embodiment, the HEV infection is of genotype 1, genotype 2 or genotype 3. In yet another embodiment, the subject is a pregnant woman, an immune-compromised subject or immune-deficient subject.

### DETAILED DESCRIPTION

Provided herein are methods of ameliorating and/or treating a Hepatitis E (HEV) infection, as well as compounds for use in such treatment. In an aspect, provided herein are compounds of Formula (I) which can be used for treatment of Hepatitis E viral infections. Also provided herein are pharmaceutical compositions comprising compounds of Formula (I).

### Definitions

Listed below are definitions of various terms used to describe this present disclosure. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the applicable art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include,” “includes,” and “included,” is not limiting.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

All ranges disclosed herein are inclusive of the recited endpoint and independently combinable (for example, the range of “from 50 mg to 300 mg” is inclusive of the endpoints, 50 mg and 300 mg, and all the intermediate values). The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. In at least some instances, the approximating language can correspond to the precision of an instrument for measuring the value.

The term “alkyl” refers to a straight- or branched-chain alkyl group having from 1 to 12 carbon atoms in the chain. Examples of alkyl groups include methyl (Me, which also may be structurally depicted by the symbol, “/”), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. The term C₁₋₄alkyl as used here refers to a straight- or branched-chain alkyl group having from 1 to 4 carbon atoms in the chain. The term C₁₋₆alkyl as used here refers to a straight- or branched-chain alkyl group having from 1 to 6 carbon atoms in the chain.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

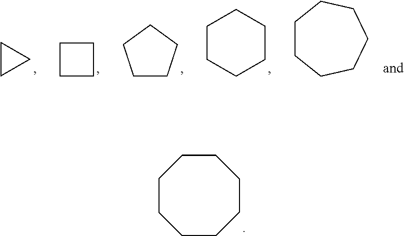

A monocyclic, bicyclic or tricyclic aromatic carbocycle represents an aromatic ring system consisting of 1, 2 or 3 rings, said ring system being composed of only carbon atoms; the term aromatic is well known to a person skilled in the art and designates cyclically conjugated systems of 4n+2 electrons, that is with 6, 10, 14 etc. π-electrons (rule of Hückel).

Particular examples of monocyclic, bicyclic or tricyclic aromatic carbocycles are phenyl, naphthalenyl, anthracenyl.

The term “phenyl” represents the following moiety:

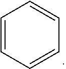

The term “heteroaryl” refers to an aromatic monocyclic or bicyclic aromatic ring system having 5 to 10 ring members and which contains carbon atoms and from 1 to 4 heteroatoms independently selected from the group consisting of N, O, and S. Included within the term heteroaryl are aromatic rings of 5 or 6 members wherein the ring consists of carbon atoms and has at least one heteroatom member. Suitable heteroatoms include nitrogen, oxygen, and sulfur. In the case of 5 membered rings, the heteroaryl ring preferably contains one member of nitrogen, oxygen or sulfur and, in addition, up to 3 additional nitrogens. In the case of 6 membered rings, the heteroaryl ring preferably contains from 1 to 3 nitrogen atoms. For the case wherein the 6 membered ring has 3 nitrogens, at most 2 nitrogen atoms are adjacent. Examples of heteroaryl groups include furyl, thienyl, pyrrolyl, oxazolyl, thiazolyl, imidazolyl, pyrazolyl, oxazolyl, thiazolyl, oxadiazolyl, triazolyl, thiadiazolyl, pyridinyl, pyridazinyl, pyrimidinyl, pyrazinyl, indolyl, isoindolyl, benzofuryl, benzothienyl, indazolyl, benzimidazolyl, benzothiazolyl, benzoxazolyl, benzisoxazolyl, benzothiadiazolyl, benzotriazolyl, quinolinyl, isoquinolinyl and quinazolinyl. Unless otherwise noted, the heteroaryl is attached to its pendant group at any heteroatom or carbon atom that results in a stable structure.

Those skilled in the art will recognize that the species of heteroaryl groups listed or illustrated above are not exhaustive, and that additional species within the scope of these defined terms may also be selected.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

The terms “buffered” solution or “buffer” solution are used herein interchangeably according to their standard meaning. Buffered solutions are used to control the pH of a medium, and their choice, use, and function is known to those of ordinary skill in the art. See, for example, G. D. Considine, ed., Van Nostrand's Encyclopedia of Chemistry, p. 261, 5^{th} ed. (2005), describing, inter alia, buffer solutions and how the concentrations of the buffer constituents relate to the pH of the buffer. For example, a buffered solution is obtained by adding MgSO₄ and NaHCO₃ to a solution in a 10:1 w/w ratio to maintain the pH of the solution at about 7.5.

Any formula given herein is intended to represent compounds having structures depicted by the structural formula as well as certain variations or forms. In particular, compounds of any formula given herein may have asymmetric centers and therefore exist in different enantiomeric forms. All optical isomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers.

It is also to be understood that compounds that have the same molecular formula but differ in the nature or sequence of bonding of their atoms or the arrangement of their atoms in space are termed “isomers.”

Stereoisomers that are not mirror images of one another are termed “diastereomers” and those that are non-superimposable mirror images of each other are termed “enantiomers.” When a compound has an asymmetric center, for example, it is bonded to four different groups, and a pair of enantiomers is possible. An enantiomer can be characterized by the absolute configuration of its asymmetric center and is described by the R-and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either an individual enantiomer or as a mixture thereof. A mixture containing equal proportions of the enantiomers is called a “racemic mixture.”

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci-and nitro-forms of phenyl nitromethane, that are likewise formed by treatment with acid or base. For example, all tautomers of a phosphate and a phosphorothioate groups are intended to be included. Examples of tautomers of a phosphorothioate include the following:

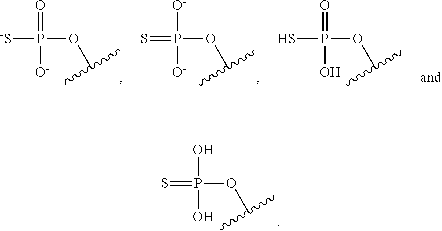

Furthermore, all tautomers of heterocyclic bases known in the art are intended to be included, including tautomers of natural and non-natural purine-bases and pyrimidine-bases.

Tautomeric forms may be relevant to the attainment of the optimal chemical reactivity and biological activity of a compound of interest.

The compounds of this present disclosure may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

Certain examples contain chemical structures that are depicted as an absolute enantiomer but are intended to indicate enantiopure material that is of unknown configuration. In these cases (R*) or (S*) or (*R) or (*S) is used in the name to indicate that the absolute stereochemistry of the corresponding stereocenter is unknown. Thus, a compound designated as (R*) or (*R) refers to an enantiopure compound with an absolute configuration of either (R) or (S). In cases where the absolute stereochemistry has been confirmed, the structures are named using (R) and (S).

The symbols [symbol] and [symbol] are used as meaning the same spatial arrangement in chemical structures shown herein. Analogously, the symbols [symbol] and [symbol] are used as meaning the same spatial arrangement in chemical structures shown herein.

Additionally, any formula given herein is intended to refer also to hydrates, solvates, and polymorphs of such compounds, and mixtures thereof, even if such forms are not listed explicitly. Certain compounds of Formula (I), or pharmaceutically acceptable salts of compounds of Formula (I), may be obtained as solvates. Solvates include those formed from the interaction or complexation of compounds of the present disclosure with one or more solvents, either in solution or as a solid or crystalline form. In some embodiments, the solvent is water and the solvates are hydrates. In addition, certain crystalline forms of compounds of Formula (I), or pharmaceutically acceptable salts of compounds of Formula (I) may be obtained as co-crystals. In certain embodiments of the present disclosure, compounds of Formula (I) were obtained in a crystalline form. In other embodiments, crystalline forms of compounds of Formula (I) were cubic in nature. In other embodiments, pharmaceutically acceptable salts of compounds of Formula (I) were obtained in a crystalline form. In still other embodiments, compounds of Formula (I) were obtained in one of several polymorphic forms, as a mixture of crystalline forms, as a polymorphic form, or as an amorphous form. In other embodiments, compounds of Formula (I) convert in solution between one or more crystalline forms and/or polymorphic forms.

Reference to a compound herein stands for a reference to any one of: (a) the actually recited form of such compound, and (b) any of the forms of such compound in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH_{(aq)} and/or R—COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

In another example, a zwitterionic compound is encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEBI:27369 by the Chemical Entities of Biological Interest (ChEBI) dictionary of molecular entities. As generally well known, a zwitterion or zwitterionic compound is a neutral compound that has formal unit charges of opposite sign. Sometimes these compounds are referred to by the term “inner salts.” Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterion ⁺H₃NCH₂COO⁻. Zwitterions, zwitterionic compounds, inner salts and dipolar ions in the known and well established meanings of these terms are within the scope of this present disclosure, as would in any case be so appreciated by those of ordinary skill in the art. Because there is no need to name each and every embodiment that would be recognized by those of ordinary skill in the art, no structures of the zwitterionic compounds that are associated with the compounds of this present disclosure are given explicitly herein. They are, however, part of the embodiments of this present disclosure. No further examples in this regard are provided herein because the interactions and transformations in a given medium that lead to the various forms of a given compound are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorus, sulfur, fluorine, chlorine, and iodine such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, ¹²⁵I, respectively. Such isotopically labeled compounds are useful in metabolic studies (preferably with ¹⁴C), reaction kinetic studies (with, for example deuterium (i.e., D or ²H); or tritium (i.e., T or ³H)), detection or imaging techniques such as positron emission tomography (PET) or single-photon emission computed tomography (SPECT) including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this present disclosure and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this present disclosure given according to the choices S¹ₑₓₐₘₚₗₑ is S₁ and S²example is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein. The foregoing convention given herein for substituents extends, when applicable, to members such as R¹, R², R³, R⁴, R⁵, G¹, G², G³, G⁴, G⁵, G⁶, G⁷, G⁸, G⁹, G¹⁰, G¹¹, n, L, R, T, Q, W, X, Y, and Z and any other generic substituent symbol used herein.

Furthermore, when more than one assignment is given for any member or substituent, embodiments of this present disclosure comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this present disclosure for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein. The foregoing convention given herein for substituents extends, when applicable, to members such as R¹, R², R³, R⁴, R⁵, G¹, G², G³, G⁴, G⁵, G⁶, G⁷, G⁸, G⁹, G¹⁰, G¹¹, n, L, R, T, Q, W, X, Y, and Z and any other generic substituent symbol used herein.

The nomenclature “Cᵢ₋ⱼ” with j>i, when applied herein to a class of substituents, is meant to refer to embodiments of this present disclosure for which each and every one of the number of carbon members, from i to j including i and j, is independently realized. By way of example, the term C₁₋₄ refers independently to embodiments that have one carbon member (C₁), embodiments that have two carbon members (C₂), embodiments that have three carbon members (C₃), and embodiments that have four carbon members (C₄).

The term Cₙ₋ₘalkyl refers to an aliphatic chain, whether straight or branched, with a total number N of carbon members in the chain that satisfies n≤N≤m, with m>n. Any disubstituent referred to herein is meant to encompass the various attachment possibilities when more than one of such possibilities are allowed. For example, reference to disubstituent —A—B—, where A≠B, refers herein to such disubstituent with A attached to a first substituted member and B attached to a second substituted member, and it also refers to such disubstituent with A attached to the second substituted member and B attached to the first substituted member.

The present disclosure includes also pharmaceutically acceptable salts of the compounds of Formula (I), preferably of those described above and of the specific compounds exemplified herein, and methods of treatment using such salts.

The term “pharmaceutically acceptable” means approved or approvable by a regulatory agency of Federal or a state government or the corresponding agency in countries other than the United States, or that is listed in the U. S. Pharmcopoeia or other generally recognized pharmacopoeia for use in animals, and more particularly, in humans.

A “pharmaceutically acceptable salt” is intended to mean a salt of a free acid or base of compounds represented by Formula (I) that are non-toxic, biologically tolerable, or otherwise biologically suitable for administration to the subject. It should possess the desired pharmacological activity of the parent compound. See, generally, G. S. Paulekuhn, et al., “Trends in Active Pharmaceutical Ingredient Salt Selection based on Analysis of the Orange Book Database”, J. Med. Chem., 2007, 50:6665-72, S. M. Berge, et al., “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt.

The present disclosure also relates to pharmaceutically acceptable prodrugs of the compounds of Formula (I), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The present disclosure also relates to pharmaceutically active metabolites of the compounds of Formula (I), which may also be used in the methods of the present disclosure. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini, et al., J Med Chem. 1997, 40, 2011-2016; Shan, et al., J Pharm Sci. 1997, 86 (7), 765-767; Bagshawe, Drug Dev Res. 1995, 34, 220-230; Bodor, Adv Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

As used herein, the term “composition” or “pharmaceutical composition” refers to a mixture of at least one compound provided herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound provided herein within or to the patient such that it can perform its intended function. Typically, such constructs are carried or transported from one organ, or portion of the body, to another organ, or portion of the body. Each carrier must be “acceptable” in the sense of being compatible with the other ingredients of the formulation, including the compound provided herein, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound provided herein, and are physiologically acceptable to the patient. Supplementary active compounds can also be incorporated into the compositions. The “pharmaceutically acceptable carrier” can further include a pharmaceutically acceptable salt of the compound provided herein. Other additional ingredients that can be included in the pharmaceutical compositions provided herein are known in the art and described, for example in Remington's Pharmaceutical Sciences (Genaro, Ed., Mack Publishing Co., 1985, Easton, Pa.), which is incorporated herein by reference.

As used herein, the term “physiologically acceptable” refers to a carrier, diluent or excipient that does not abrogate the biological activity and properties of the compound.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The term “stabilizer,” as used herein, refers to polymers capable of chemically inhibiting or preventing degradation of a compound of Formula I. Stabilizers are added to formulations of compounds to improve chemical and physical stability of the compound.

The term “tablet,” as used herein, denotes an orally administrable, single-dose, solid dosage form that can be produced by compressing a drug substance or a pharmaceutically acceptable salt thereof, with suitable excipients (e.g., fillers, disintegrants, lubricants, glidants, and/or surfactants) by conventional tableting processes. The tablet can be produced using conventional granulation methods, for example, wet or dry granulation, with optional comminution of the granules with subsequent compression and optional coating. The tablet can also be produced by spray-drying.

As used herein, the term “capsule” refers to a solid dosage form in which the drug is enclosed within either a hard or soft soluble container or “shell.” The container or shell can be formed from gelatin, starch and/or other suitable substances.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

The term “combination,” “therapeutic combination,” “pharmaceutical combination,” or “combination product” as used herein refer to a non-fixed combination or a kit of parts for the combined administration where two or more therapeutic agents can be administered independently, at the same time or separately within time intervals, especially where these time intervals allow that the combination partners show a cooperative, e.g., synergistic, effect.

The term “modulators” include both inhibitors and activators, where “inhibitors” refer to compounds that decrease, prevent, inactivate, desensitize, or down-regulate HEV assembly and other HEV core protein functions necessary for HEV replication or the generation of infectious particles.

As used herein, the term “treatment” or “treating,” is defined as the application or administration of a therapeutic agent, i.e., a compound of the present disclosure (alone or in combination with another pharmaceutical agent), to a patient, or application or administration of a therapeutic agent to an isolated tissue or cell line from a patient (e.g., for diagnosis or ex vivo applications), who has an HEV infection, a symptom of HEV infection or the potential to develop an HEV infection, with the purpose to cure, heal, alleviate, relieve, alter, remedy, ameliorate, improve or affect the HEV infection, the symptoms of HEV infection or the potential to develop an HEV infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject or individual is human.

In treatment methods according to the present disclosure, an effective amount of a pharmaceutical agent according to the present disclosure is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the compounds of the present disclosure may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID, and, in an embodiment, BID). An example of a dose is in the range of from about 10 to about 300 mg of compound per kg of subject's body weight per day, in an embodiment about 15 to 250 mg/kg/day, or about 20 to 200 mg/kg/day, in single or divided dosage units (e.g., BID, TID, QID, and, in an embodiment, BID). A high dose may be about 200 mg/kg/day, while a medium dose may be about 70 mg/kg/day and a low dose may be about 20 mg/kg/day. For a 70-kg human, an illustrative range for a suitable dosage amount is from about 0.05 to about 7 g/day, or about 0.2 to about 2.5 g/day.

An example of a dose of a compound is from about 1 mg to about 2,500 mg. In some embodiments, a dose of a compound of the present disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., another drug for HEV treatment) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

### Compounds

In an aspect, provided herein are compounds of formula (I)

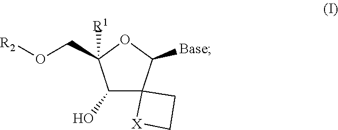

or a pharmaceutically acceptable salt thereof;

wherein:

Base is selected from the group consisting of (b-1), (b-2), (b-3), (b-4), (b-5), (b-6), (b-7), and (b-8):

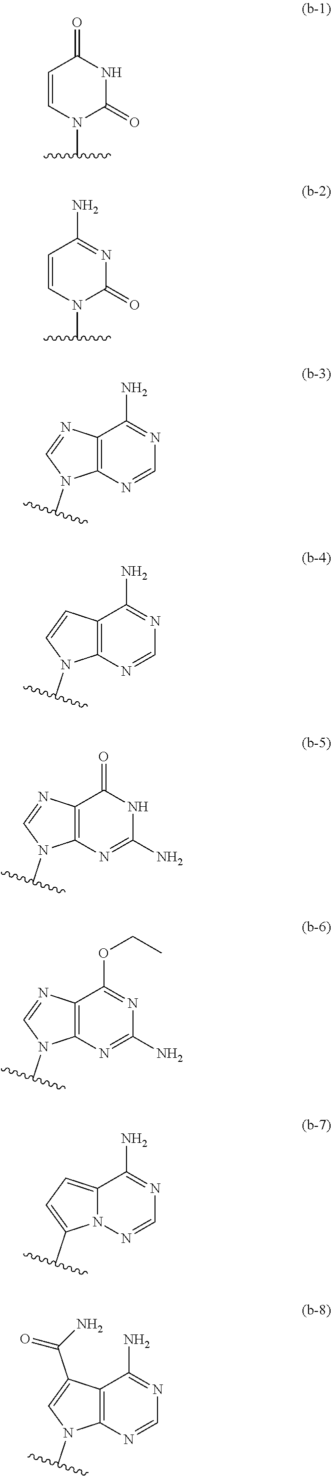

X is selected from the group consisting of O and S;

R¹ is selected from the group consisting of H, F, and N₃; and

R² is selected from the group consisting of (f-1) and (f-2):

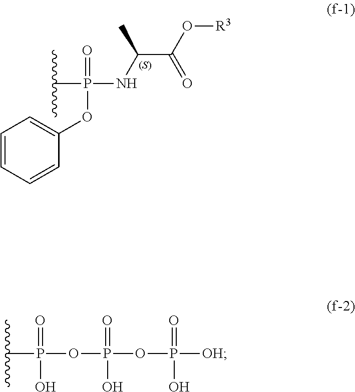

and

R³ is C₁₋₄alkyl.

In an embodiment, Base is (b-1). In another embodiment, R² is (f-1). In yet another embodiment, Base is (b-1), X is S, R² is (f-1) and R³ is isopropyl. In still another embodiment, Base is (b-1), X is O, R² is (f-1) and R³ is butyl.

In an embodiment, the compound is selected from the group consisting of:

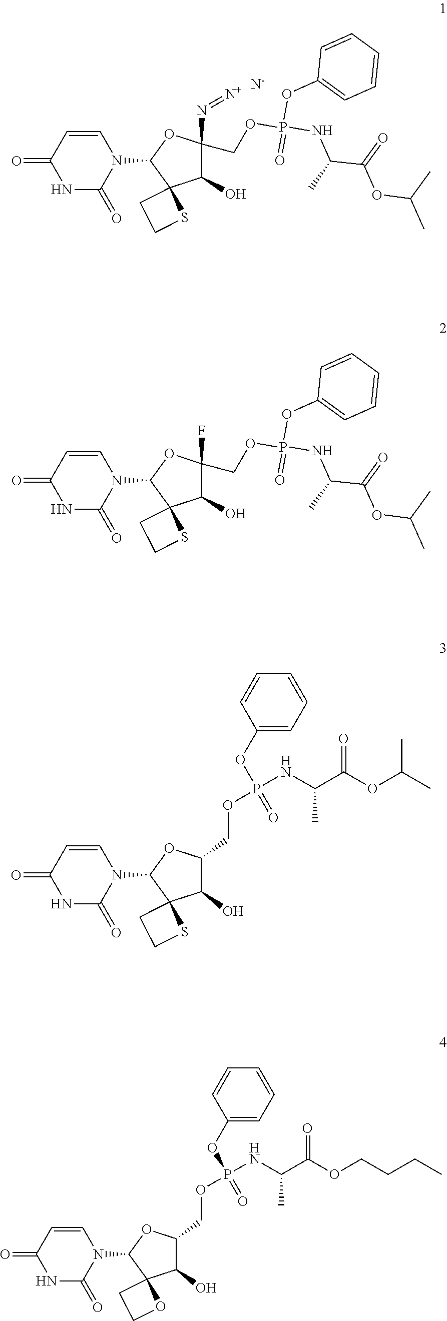

or a pharmaceutically acceptable salt thereof, more particularly compound 1 or a pharmaceutically acceptable salt thereof.

Compounds of the Formula (I) may be prepared by methods known to those skilled in the art and/or by variants of such methods using routine experimentation guided by the teachings provided herein.

### Pharmaceutical Compositions

Also provided herein are pharmaceutical compositions comprising at least one compound of Formula I and at least one pharmaceutically acceptable excipient.

Some embodiments described herein relate to the use of a pharmaceutical composition, that can include an effective amount of one or more compounds described herein (e.g., a compound of Formula (I), or a pharmaceutically acceptable salt thereof) and a pharmaceutically acceptable carrier, diluent, excipient or combination thereof.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen.

Techniques for formulation and administration of the compounds described herein are known to those skilled in the art.

As used herein, the term “composition” or “pharmaceutical composition” refers to a mixture of at least one compound useful within the present disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the present disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the present disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure and are physiologically acceptable to the patient. Supplementary active compounds may also be incorporated into the compositions. The “pharmaceutically acceptable carrier” may further include a pharmaceutically acceptable salt of the compound useful within the present disclosure. Other additional ingredients that may be included in the pharmaceutical compositions used in the practice of the present disclosure are known in the art and described, for example in Remington's Pharmaceutical Sciences (Genaro, Ed., Mack Publishing Co., 1985, Easton, Pa.), which is incorporated herein by reference.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the present disclosure can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Oral tablets may include a compound according to the present disclosure mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract or may be coated with an enteric coating.

Capsules for oral administration include hard and soft gelatin capsules. To prepare hard gelatin capsules, compounds of the present disclosure may be mixed with a solid, semi-solid, or liquid diluent. Soft gelatin capsules may be prepared by mixing the compound of the present disclosure with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this present disclosure may also be administered by non-oral routes. For example, the compositions may be formulated for rectal administration as a suppository. For parenteral use, including intravenous, intramuscular, intraperitoneal, or subcutaneous routes, the compounds of the present disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the present disclosure may utilize a patch formulation to affect transdermal delivery.

Compounds of the present disclosure may alternatively be administered in methods of this present disclosure by inhalation, via the nasal or oral routes, e.g., in a spray formulation also containing a suitable carrier.

Methods of Treatment Provided herein are methods of ameliorating and/or treating a HEV infection, which can include administering to a subject in need thereof an effective amount of one or more compounds and/or a pharmaceutically acceptable salt thereof as described herein, or a pharmaceutical composition that includes one or more compounds and/or a pharmaceutically acceptable salt thereof as described herein, wherein the compounds and their pharmaceutically acceptable salts described herein can be of Formula (I), or a pharmaceutically acceptable salt thereof.

Other embodiments described herein relate to a compound and/or a pharmaceutically acceptable salt thereof as described herein, for use in ameliorating or treating a HEV infection, or a pharmaceutical composition for use in ameliorating or treating a HEV infection as described herein that includes one or more compounds and/or a pharmaceutically acceptable salt thereof as described herein, wherein the compounds and their pharmaceutically acceptable salts described herein can be of Formula (I), or a pharmaceutically acceptable salt thereof.

Other embodiments described herein relate to a method of inhibiting viral replication of a HEV, which can include contacting a cell infected with the HEV with an effective amount of a compound described herein (e.g., of Formula (I), or a pharmaceutically acceptable salt thereof), and/or a pharmaceutical composition that includes one or more compounds described herein (e.g., a compound of Formula (I), or a pharmaceutically acceptable salt thereof). In an embodiment, the method of inhibiting viral replication of a HEV is an in vitro method.

In some embodiments, an effective amount of one or more compounds described herein (e.g., a compound of Formula (I), or a pharmaceutically acceptable salt thereof), and/or a pharmaceutical composition that includes one or more compounds described herein (e.g., a compound of Formula (I), or a pharmaceutically acceptable salt thereof) can be used to treat ameliorate and/or prevent one more symptoms of an infection caused by a HEV (e.g., by administration to a subject in need thereof). For example, a compound of Formula (I), or a pharmaceutically acceptable salt thereof, can be used to treat, ameliorate and/or prevent one or more of the following symptoms caused by a HEV infection: fever, jaundice, educed appetite (anorexia), nausea, vomiting, abdominal pain, itching, skin rash, and/or joint pain.

In some embodiments, an effective amount of one or more compounds described herein (e.g., a compound of Formula (I), or a pharmaceutically acceptable salt thereof), and/or a pharmaceutical composition that includes one or more compounds described herein (e.g., a compound of Formula (I), or a pharmaceutically acceptable salt thereof) can be used to treat, ameliorate and/or prevent one more conditions related to an infection caused by a HEV (e.g., by administration of an effective amount to a subject in need thereof). For example, in an embodiment, an effective amount of a compound of Formula (I), or a pharmaceutically acceptable salt thereof, can be used to slow or prevent the progression of a HEV infection to a chronic HEV infection in a subject to which the compound or salt is administered. In an embodiment, an effective amount of a compound of Formula (I), or a pharmaceutically acceptable salt thereof, can be used to ameliorate or treat a HEV-associated disease or HEV-induced disease (e.g., a chronic HEV-induced disease) in a subject to which the compound or salt is administered. In an embodiment, an effective amount of a compound of Formula (I), or a pharmaceutically acceptable salt thereof, can be used to slow or prevent the aggravation of a HEV-associated disease or HEV-induced disease (e.g., chronic HEV-induced disease) in a subject to which the compound or salt is administered. Examples of such HEV-associated diseases or HEV-induced (chronic) diseases include acute pancreatitis, fulminant liver failure, Guillain-Barré syndrome, neuralgic amyotrophy, hemolytic anemia (e.g., in a subject with G6PD deficiency), glomerulonephritis, glomerulonephritis with nephrotic syndrome, cryoglobulinemia, mixed cryoglobulinemia, and/or thrombocytopenia.

In some embodiments, an effective amount of one or more compounds described herein (e.g., a compound of Formula (I), or a pharmaceutically acceptable salt thereof), and/or a pharmaceutical composition that includes one or more compounds described herein (e.g., a compound of Formula (I), or a pharmaceutically acceptable salt thereof) can be used to treat, ameliorate and/or prevent one more fibrotic or fibrotic-related conditions that are related to an infection caused by a HEV (e.g., by administration of an effective amount to a subject in need thereof). In an embodiment, an effective amount of a compound of Formula (I), or a pharmaceutically acceptable salt thereof, can be used to ameliorate (e.g., slow or prevent the progression of) a stage of fibrosis in a subject having an HEV infection to which the compound or salt is administered. For example, an effective amount of a compound of Formula (I), or a pharmaceutically acceptable salt thereof, can be used to ameliorate the extent of liver damage in a subject having an HEV infection to which the compound or salt is administered, wherein the liver damage is caused or aggravated by HEV infection (including chronic HEV infection). In another embodiment, an effective amount of a compound of Formula (I), or a pharmaceutically acceptable salt thereof, can be used to ameliorate fibrosis (e.g., slow or prevent the progression of fibrosis) in a subject having an HEV infection to which the compound or salt is administered. For example, in an embodiment, an effective amount of a compound of Formula (I), or a pharmaceutically acceptable salt thereof, can be used to prevent cirrhosis (e.g., slow or prevent the progression of an earlier stage of liver fibrosis to a cirrhotic stage) in a subject having an HEV infection (including chronic HEV infection) to which the compound or salt is administered.

In some embodiments, the particular characteristics of a subject are taken into consideration when using a compound (or pharmaceutically acceptable salt thereof) or carrying out a method as described herein. In addition to being identified as a subject in need of treatment for a condition as described herein (such as a HEV infection), a subject can also be identified on the basis of a particular characteristic that results in vulnerability to HEV infection or its effects. For example, in an embodiment, the subject has hemolytic anemia and also has the hereditary risk factor glucose-6-phosphate dehydrogenase deficiency (G6PD deficiency). In various embodiments the subject can be in need of treatment for a condition as described herein (such as a HEV infection) and also a pregnant woman, an immuno-compromised subject, an immuno-deficient subject and/or organ transplant patient. Thus, any of the compound or salt administration steps of the methods described herein can be conducted in conjunction with a step of identifying one or more clinically relevant characteristics of the subject. For example, an embodiment provides a method of ameliorating or treating a Hepatitis E (HEV) infection comprising identifying a pregnant woman subject in need thereof and administering to the subject an amount of a compound of Formula (I), or a pharmaceutically acceptable salt thereof that is effective to treat the HEV infection and thereby prevent or slow progression to fulminant liver failure.

In some embodiments, the particular characteristics of the HEV are taken into consideration when using a compound (or pharmaceutically acceptable salt thereof) or carrying out a method as described herein. For example, as noted above, HEV can be genotype 1, genotype 2, genotype 3, or genotype 4, with various known subtypes. In addition to being identified as a subject in need of treatment for a condition as described herein (such as a HEV infection), a subject can also be identified on the basis of a particular characteristic of the HEV itself, such as genotype.

In some embodiments, the particular characteristics of the compound (or salt) described herein are taken into consideration when using the compound or carrying out a method as described herein. For example, the compounds of Formula (I), and their pharmaceutically acceptable salts can have various potencies. In an embodiment, a compound of Formula (I), or a pharmaceutically acceptable salt thereof, has an EC₅₀ of 0.30 μM or less; an EC₅₀ of 0.25 μM or less, an EC₅₀ of 0.20 μM or less; or an EC₅₀ μM of 0.15 or less. In an embodiment, a compound of Formula (I), or a pharmaceutically acceptable salt thereof, has an EC₅₀ of about 75 μM or less; an EC₅₀ of about 50 μM or less, an EC₅₀ of about 30 μM or less; an EC₅₀ of about 10 μM or less; an EC₅₀ of about 5 μM or less; or an EC₅₀ of about 1 μM or less. Potency data for exemplified embodiments of compounds of Formula (I) are provided in the Examples below. The application relates more particularly to those compounds as defined herein which show an EC₅₀ of less than 0.30 μM (more particularly of 0.25 μM or less, or of 0.20 μM or less, or of 0.15 μM or less) for the inhibition of HEV DNA for example in the Huh7 cell line (eg as described in example 2 below), more particularly an EC₅₀ of less than 0.30 μM (more particularly of 0.25 μM or less, or of 0.20 μM or less, or of 0.15 μM or less) for the inhibition of HEV DNA when measured 3 days after the compound has been placed in the Huh7 cell culture (eg as described in example 2 below). As used herein, the half maximal effective concentration (EC50) is intended in accordance with its general meaning in the field. It may more particularly refer to the concentration of a compound which induces a response halfway between the baseline and maximum, typically after a specified exposure time. The EC50 value is commonly used as a measure of a compound's potency, with a lower value generally indicating a higher potency.

Various indicators for determining the effectiveness of a method for treating a viral infection, such as a HEV infection, are known to those skilled in the art. Example of suitable indicators include, but are not limited to, a reduction in viral load, a reduction in viral replication, a reduction in time to seroconversion (virus undetectable in patient serum), a reduction of morbidity or mortality in clinical outcomes, and/or other indicator of disease response.

In some embodiments, an effective amount of a compound of Formula (I), or a pharmaceutically acceptable salt thereof, is an amount that is effective to reduce viral titers to undetectable levels, for example, to about 1000 to about 5000, to about 500 to about 1000, or to about 100 to about 500 genome copies/mL serum. In some embodiments, an effective amount of a compound of Formula (I), or a pharmaceutically acceptable salt thereof, is an amount that is effective to reduce viral load compared to the viral load before administration of the compound of Formula (I), or a pharmaceutically acceptable salt thereof. In some embodiments, an effective amount of a compound of Formula (I), or a pharmaceutically acceptable salt thereof, is an amount that is effective to achieve a reduction in viral titer in the serum of the subject in the range of about 1.5-log to about a 2.5-log reduction, about a 3-log to about a 4-log reduction, or a greater than about 5-log reduction compared to the viral load before administration of the compound of Formula (I), or a pharmaceutically acceptable salt thereof. For example, in an embodiment the viral load is measured before administration of the compound of Formula (I), or a pharmaceutically acceptable salt thereof, and again after completion of the treatment regime with the compound of Formula (I), or a pharmaceutically acceptable salt thereof (for example, 1 week after completion). In some embodiments, a compound of Formula (I), or a pharmaceutically acceptable salt thereof, can result in at least a 1, 2, 3, 4, 5, 10, 15, 20, 25, 50, 75, 100-fold or more reduction in the replication of a HEV relative to pre-treatment levels in a subject, as determined after completion of the treatment regime (for example, 1 week after completion).

In some embodiments, a compound of Formula (I), or a pharmaceutically acceptable salt thereof, can result in a reduction of the replication of a HEV relative to pre-treatment levels in the range of about 2 to about 5 fold, about 10 to about 20 fold, about 15 to about 40 fold, or about 50 to about 100 fold.

As will be readily apparent to one skilled in the art, the useful in vivo dosage to be administered and the particular mode of administration will vary depending upon the age, weight, the severity of the affliction, and mammalian species treated, the particular compounds or pharmaceutically acceptable salts thereof employed, and the specific use for which these compounds or salts are employed. The determination of effective dosage levels, that is the dosage levels necessary to achieve the desired result, can be accomplished by one skilled in the art using routine methods, for example, human clinical trials and in vitro studies.

The dosage may range broadly, depending upon the desired effects and the therapeutic indication. Alternatively, dosages may be based and calculated upon the surface area of the patient, as understood by those of skill in the art. Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the subject. In some embodiments, the compounds will be administered for a period of continuous therapy, for example for a week or more, or for months or years.

In instances where human dosages for compounds have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound or pharmaceutically acceptable salt thereof and can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Compounds and pharmaceutically acceptable salts disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### Examples

### Example 1: Synthesis of Compounds

### Compound 1

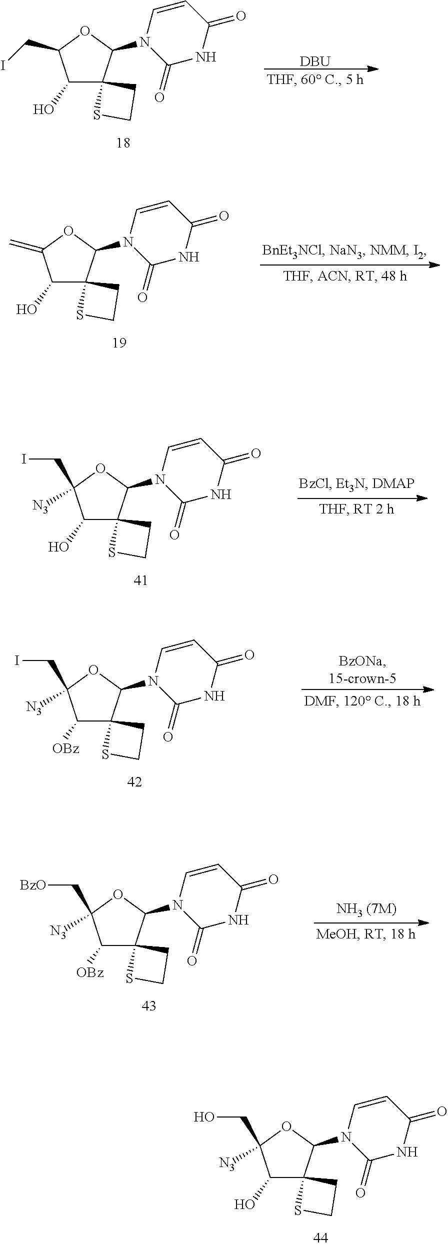

### Step 1: Synthesis of 1-((4R,5R,8R)-8-hydroxy-7-methylene-6-oxa-1-thiaspiro[3.4]octan-5-yl)pyrimidine-2,4(1H,3H)-dione 19

Intermediate 18 (5.29 g, 13.352 mmol) was solubilized in THF (150 mL) and added dropwise over 1 h to a stirred solution of DBU (3.174 mL, 1.019 g/mL, 21.245 mmol) in THF (100 mL) at 60° C. The resulting mixture was stirred at 60° C. for 5 h. The reaction mixture was allowed to cool down to RT and poured in water (200 mL). The mixture was acidified until pH=4 with 1M HCl solution. The organic layer was extracted 3 times with EtOAc (200 mL), dried over MgSO₄ and concentrated to dryness. The solid was triturated in DCM and filtrated to afford intermediate 19 (2.68 g, yield 75%) as white solid.

MS (ES-): 267.0; ¹H NMR (400 MHz, DMSO-ds) δ ppm 2.54-2.68 (m, 1H), 2.72-2.84 (m, 1H), 2.91 (td, J=8.5, 5.7 Hz, 1H), 2.94-3.05 (m, 1H), 4.26 (s, 1H), 4.45 (t, J=1.8 Hz, 1H), 4.56 (br d, J=6.2 Hz, 1H), 5.66 (d, J=7.9 Hz, 1H), 6.06 (d, J=6.4 Hz, 1H), 6.51 (s, 1H), 7.33 (d, J=8.1 Hz, 1H), 11.54 (br s, 1H).

### Step 2: Synthesis of 1-((4R,5R,7S,8R)-7-azido-8-hydroxy-7-(iodomethyl)-6-oxa-1-thiaspiro[3.4]octan-5-yl)pyrimidine-2,4(1H,3H)-dione 41

N-benzyl-N,N-diethylethanaminium Chloride (BnEt₃NCl) (4.585 g, 20.127 mmol) and sodium azide (NaN₃) (1.308 g, 20.127 mmol) were suspended in MeCN (30 mL) and stirred for 16 h. The mixture was filtrated into a solution of intermediate 19 (900 mg, 3.355 mmol) and NMM (5.4 mL, 0.917 g/mL, 48.956 mmol) in THF (60 mL). The reaction mixture was cooled to 0° C. and Iodine (5.11 g, 20.127 mmol) in THF (18 mL) was added. The reaction mixture was stirred for 5 h at RT. N-acetyl-cysteine (2 g) was added to the mixture until no gas evolved. Saturated aqueous Na₂S₂O₃ was added to the mixture until a light yellow solution developed. The solution was concentrated under reduced pressure then diluted in EtOAc (50 mL). The organic layer was washed with brine and dried over MgSO₄. Solvent was removed and the crude was purified by column chromatography using Heptane/EtOAc as eluent to afford intermediate 41 (1.49 g, yield 99%).

MS (ES-): 436.0; ¹H NMR (400 MHz, DMSO-ds) δ ppm 2.52-2.61 (m, 1H), 2.76-2.98 (m, 3H), 3.75 (s, 2H), 4.34 (br s, 1H), 5.68 (d, J=8.1 Hz, 1H), 6.47 (br d, J=6.2 Hz, 2H), 7.43-7.57 (m, 1H), 11.57 (s, 1H).

### Step 3: Synthesis of (4R,5R,7S,8R)-7-azido-5-(2,4-dioxo-3,4-dihydropyrimidin-1(2H)-yl)-7-(iodomethyl)-6-oxa-1-thiaspiro[3.4]octan-8-yl benzoate 42

Intermediate 41 (1.49 g, 3.408 mmol) was dissolved in THF (45 mL) and the mixture was cooled to 0° C. Et₃N (2.368 mL, 0.728 g/mL, 17.04 mmol) and DMAP (8.327 mg, 0.0682 mmol) were added to the mixture followed by the dropwise addition of benzoyl chloride (0.475 mL, 1.211 g/mL, 4.089 mmol). The reaction mixture was stirred for 1.5 h at RT. The reaction mixture was diluted in EtOAc (100 mL). The organic layer was washed with brine, dried over MgSO₄ and concentrated. The crude was purified by column chromatography using Heptane/EtOAc as eluent to afford intermediate 42 (1.5 g, yield 81%) as white foam.

MS (ES-): 540.0; ¹H NMR (400 MHz, DMSO-ds) δ ppm 2.73-2.84 (m, 2H), 2.84-2.94 (m, 1H), 3.02-3.12 (m, 1H), 3.79 (br d, J=11.7 Hz, 1H), 3.92 (br d, J=11.7 Hz, 1H), 5.77 (dd, J=8.0, 2.1 Hz, 1H), 6.02 (br s, 1H), 6.50 (br s, 1H), 7.63 (t, J=7.2 Hz, 2H), 7.72-7.85 (m, 2H), 8.18 (d, J=7.6 Hz, 2H), 11.63 (s, 1H).

### Step 4: Synthesis of [(4R,5R,6R,8R)-6-azido-5-benzoyloxy-8-(2,4-dioxopyrimidin-1-yl)-7-oxa-1-thiaspiro[3.4]octan-6-yl]methyl benzoate 43

Intermediate 42 (1.5 g, 2.771 mmol) and BzONa (1.997 g, 13.855 mmol) were suspended in DMF (80 mL) followed by the addition of 15-crown-5 (5.499 mL, 1.11 g/mL, 27.71 mmol). The reaction mixture was stirred overnight at 120° C. The reaction mixture was diluted in EtOAc (100 mL), filtrated over a small bed of decalite and washed with water. The organic layer was dried over MgSO₄ and the solvent was removed. The crude was purified by column chromatography using Heptane/EtOAc as eluent to afford intermediate 43 (700 mg, yield 47%) as light yellow solid 63% pure as determined by LC-MS. The compound was used as such.

MS (ES-): 534.1

### Step 5: Synthesis of (4R,5R,7R,8R)-7-azido-7-((benzoyloxy)methyl)-5-(2,4-dioxo-3,4-dihydropyrimidin-1(2H)-yl)-6-oxa-1-thiaspiro[3.4]octan-8-yl benzoate 44

Intermediate 43 (700 mg, 1.307 mmol) was dissolved in NH₃ (7M in MeOH) (150 mL, 7M, 1050 mmol) and the mixture was stirred overnight at RT. The reaction mixture was concentrated until dryness and the solid was triturated in Et₂O to afford 1-[(4R,5R,6R,8R)-6-azido-5-hydroxy-6-(hydroxymethyl)-7-oxa-1-thiaspiro[3.4]octan-8-yl]pyrimidine-2,4-dione 44 (360 mg, yield 84%) as light yellow solid

MS (ES-): 326.0; ¹H NMR (400 MHz, DMSO-ds) δ ppm 2.38-2.48 (m, 1H), 2.78-2.92 (m, 2H), 3.02-3.10 (m, 1H), 3.69-3.78 (m, 2H), 4.11 (br d, J=5.3 Hz, 1H), 5.67 (d, J=8.1 Hz, 1H), 5.76 (br s, 1H), 5.93 (br d, J=4.2 Hz, 1H), 6.60 (br s, 1H), 7.66 (d, J=8.1 Hz, 1H), 11.31 (br s, 1H).

### Synthesis of isopropyl (2R)-2-[[[(4R,5R,6R,8R)-6-azido-8-(2,4-dioxopyrimidin-1-yl)-5-hydroxy-7-oxa-1-thiaspiro[3.4]octan-6-yl]methoxy-phenoxy-phosphoryl]amino]propanoate 45

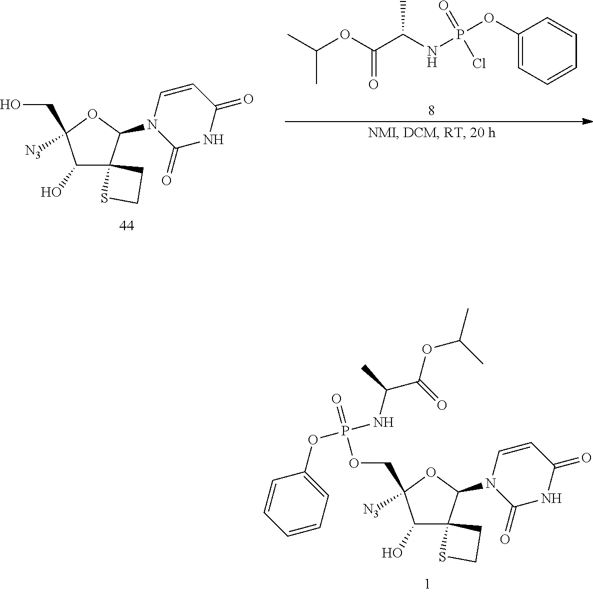 [SMILES: [N-]=[N+]=N[C@]1(CO)O[C@@H](n2ccc(=O)[nH]c2=O)[C@@]2(CCS2)[C@@H]1O ; CC(C)OC(=O)[C@H](C)NP(=O)(Cl)Oc1ccccc1 ; CC(C)OC(=O)[C@H](C)NP(=O)(OC[C@@]1(N=[N+]=[N-])O[C@@H](n2ccc(=O)[nH]c2=O)[C@@]2(CCS2)[C@@H]1O)Oc1ccccc1]

Intermediate 44 (200 mg, 0.611 mmol) was dissolved in dry pyridine (5 mL) and the solvent was removed under reduced pressure to obtain a foam. The foam was dissolved in DCM (10 mL) and (0.244 mL, 1.03 g/mL, 3.055 mmol) of N-methylimidazole was added. The reaction mixture was stirred for 5 min at RT under N₂ atmosphere. Isopropyl (2R)-2-[[chloro(phenoxy)phosphoryl]amino]propanoate 8 (1M in THF) (0.917 mL, 1 M, 0.917 mmol) was added and the reaction mixture was stirred for 20 h at RT under N₂ atmosphere. The reaction mixture was poured in cold water (20 mL) and DCM (20 mL). The aqueous layer was extracted with DCM (3×50 mL). The organic layer was dried over MgSO₄ and the solvent was removed under reduced pressure. The crude obtained was purified by Prep HPLC using method E. The obtained fraction was freeze-dried to deliver 1 (80 mg, yield 22%).

MS (ES-): 595.2; ¹H NMR (400 MHz, DMSO-ds) δ ppm 1.14 (dd, J=6.2, 2.4 Hz, 6H), 1.22 (d, J=7.3 Hz, 3H), 2.52-2.60 (m, 1H), 2.80-2.90 (m, 2H), 2.95-3.04 (m, 1H), 3.72-3.84 (m, 1H), 4.21-4.38 (m, 3H), 4.84 (quind, J=6.3, 6.3, 6.3, 6.3, 4.0 Hz, 1H), 5.60 (dd, J=7.9, 3.3 Hz, 1H), 6.06-6.22 (m, 2H), 6.49-6.62 (m, 1H), 7.15-7.24 (m, 3H), 7.33-7.40 (m, 2H), 7.47 (br d, J=7.0 Hz, 1H), 11.52 (br s, 1H).

### Compound 2

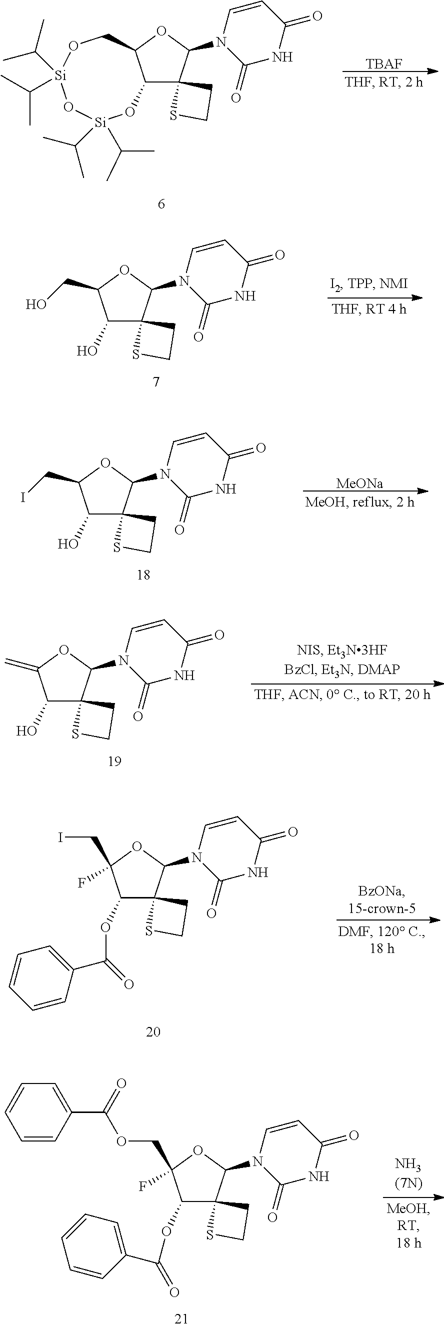

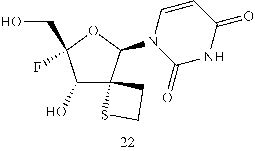 [SMILES: O=c1ccn([C@@H]2O[C@](F)(CO)[C@@H](O)[C@]23CCS3)c(=O)[nH]1]

### Step 1: Synthesis of 1-((4R,5R,7R,8R)-8-hydroxy-7-(hydroxymethyl)-6-oxa-1-thiaspiro-[3.4]octan-5-yl)pyrimidine-2,4(1H,3H)-dione 7

To a solution of intermediate 6 (15 g, 28.365 mmol) in THF (300 mL), TBAF (56.7 mL, 56.7 mmol, 1M in THF) was added. The resulting mixture was stirred under N₂ atmosphere at RT for 2 h. Afterwards, the solvent was evaporated and the crude was purified by Prep HPLC using method A to yield intermediate 7 (7 g, 86%) as white powder.

MS (ES-): 285.0; ¹H NMR (400 MHz, DMSO-ds) δ ppm 2.44-2.49 (m, 1H), 2.78-2.90 (m, 2H), 2.99-3.09 (m, 1H), 3.39-3.45 (m, 1H), 3.59 (dd, J=12.4, 2.8 Hz, 1H), 3.74 (dd, J=12.4, 2.1 Hz, 1H), 3.92 (br d, J=8.1 Hz, 1H), 5.23 (br s, 1H), 5.62 (d, J=8.1 Hz, 1H), 5.68 (br s, 1H), 6.40 (s, 1H), 8.00 (d, J=8.1 Hz, 1H), 11.40 (br s, 1H).

### Step 2: Synthesis of 1-((4R,5R,7S,8R)-8-hydroxy-7-(iodomethyl)-6-oxa-1-thiaspiro[3.4]octan-5-yl)pyrimidine-2,4(1H,3H)-dione 18

Iodine (6.649 g, 26.196 mmol) and TPP (6.871 g, 26.196 mmol) were added to a suspension of intermediate 7 (5 g, 17.464 mmol) in NMI (6.96 mL, 1.03 g/mL, 87.318 mmol) and THF (200 mL, 0.886 g/mL, 2457.462 mmol) at RT. The reaction mixture was stirred for 4 h under N₂ atmosphere. The reaction mixture was quenched with a saturated solution of Na₂S₂O₃, concentrated and diluted with EtOAc (100 mL). The organic layer was washed with brine (50 mL) dried over MgSO₄ and concentrated. The crude was purified by chromatography column using Heptane/EtOAc as eluent to afford a white solid (6 g) containing intermediate 18 80% and triphenylphosphine oxide 20%.

MS (ES-): 395.0; ¹H NMR (400 MHz, DMSO-ds) δ ppm 2.55-2.67 (m, 1H), 2.68-2.80 (m, 1H), 2.85-2.95 (m, 2H), 3.35-3.47 (m, 2H), 3.50-3.60 (m, 1H), 3.81 (t, J=6.7 Hz, 1H), 5.66 (d, J=8.1 Hz, 1H), 5.97 (d, J=6.2 Hz, 1H), 6.33 (s, 1H), 7.53 (d, J=8.1 Hz, 1H), 11.50 (s, 1H).

### Step 3: 1-((4R,5R,8R)-8-hydroxy-7-methylene-6-oxa-1-thiaspiro[3.4]octan-5-yl)pyrimidine-2,4(1H,3H)-dione 19

The mixture containing intermediate 18 (6 g) was suspended in MeOH (100 mL). NaOMe (30% in MeOH) (14.022 mL, 5.4 M, 75.718 mmol) was added to the suspension. The resulting mixture was stirred at reflux for 2.5 h. The reaction mixture was allowed to cool down to RT and filtrated over a small pad of Decalite®. The filtrate was purified by prep HPLC using method A. The fractions were freeze-dried to deliver intermediate 19 (2.6 g, 55% for two steps) as white solid.

MS (ES-): 267.0

¹H NMR (400 MHz, DMSO-d6) δ ppm 2.54-2.68 (m, 1H), 2.72-2.84 (m, 1H), 2.91 (td, J=8.5, 5.7 Hz, 1H), 2.94-3.05 (m, 1H), 4.26 (s, 1H), 4.45 (t, J=1.8 Hz, 1H), 4.56 (br d, J=6.2 Hz, 1H), 5.66 (d, J=7.9 Hz, 1H), 6.06 (d, J=6.4 Hz, 1H), 6.51 (s, 1H), 7.33 (d, J=8.1 Hz, 1H), 11.54 (br s, 1H).

### Step 4: Synthesis of (4R,5R,7R,8R)-5-(2,4-dioxo-3,4-dihydropyrimidin-1(2H)-yl)-7-fluoro-7-(iodomethyl)-6-oxa-1-thiaspiro[3.4]octan-8-yl benzoate 20

Intermediate 19 (1 g, 3.7 mmol) was dissolved in ACN (20 mL) and THF (30 mL), the resulting mixture was cooled to −15° C. under N₂ atmosphere then triethylamine trihydrofluoride (0.6 mL, 0.989 g/mL, 3.7 mmol) in 5 mL of ACN was added dropwise followed by the addition of NIS (1 g, 4.4 mmol). The resulting reaction mixture was stirred for 1 h at −15° C. under N₂ atmosphere. Afterwards, Et₃N (2.6 mL, 0.728 g/mL, 18.6 mmol) and DMAP (9.107 mg, 0.08 mmol) were added to the reaction mixture. The reaction mixture was diluted with 40 mL of THF followed by the dropwise addition of benzoyl chloride (0.433 mL, 1.211 g/mL, 3.7 mmol) at 0° C. The reaction mixture was allowed to warm up to RT and stirred for 3 h. The reaction mixture was diluted with EtOAc (30 mL) and successively washed with brine, a saturated solution of Na₂S₂O₃, dried over MgSO₄ and purified by column chromatography (heptane/EtOAc) to afford intermediate 20 (1.2 g, yield 62%) as light yellow solid.

MS (ES-): 516.8; ¹H NMR (400 MHz, DMSO-ds) δ ppm 2.80 (br s, 2H), 2.93 (br d, J=5.3 Hz, 1H), 3.04-3.20 (m, 1H), 3.50-3.77 (m, 2H), 5.78 (br d, J=7.7 Hz, 1H), 6.04 (br s, 1H), 6.59 (br s, 1H), 7.63 (br t, J=7.3 Hz, 2H), 7.70-7.98 (m, 1H), 8.18 (br d, J=7.3 Hz, 2H), 11.65 (br s, 1H).

### Step 5: Synthesis of ((4R,5R,7S,8R)-8-(benzoyloxy)-5-(2,4-dioxo-3,4-dihydropyrimidin-1(2H)-yl)-7-fluoro-6-oxa-1-thiaspiro[3.4]octan-7-yl)methyl benzoate 21

Intermediate 20 (1.2 g, 2.3 mmol), sodium benzoate (1.7 g, 11.6 mmol) and 15-crown-5 (4.6 mL, 1.11 g/mL, 23.2 mmol) were suspended in DMF (50 mL) under N₂ atmosphere. The reaction mixture was stirred for 18 h at 120° C. Afterwards, the reaction mixture was allowed to cool down to 45-50° C. then diluted with EtOAc (100 mL) and filtrated. The organic layer was washed successively with brine, a saturated solution of Na₂S₂O₃, and dried over Na₂SO₄. The solvent was removed, the crude was purified by column chromatography (heptane/EtOAc: 100/100 to 50/50) to afford intermediate 21 (700 mg, 59%) as light yellow solid.

MS (ES-): 511.0; ¹H NMR (400 MHz, CDCl₃) δ ppm 2.76 (br s, 1H), 2.89-2.95 (m, 1H), 3.11 (br s, 1H), 3.17-3.30 (m, 1H), 4.54 (dd, J=12.3, 5.7 Hz, 1H), 4.72 (dd, J=12.2, 8.7 Hz, 1H), 5.53-5.64 (m, 1H), 5.92 (s, 1H), 6.58-6.79 (m, 1H), 7.28 (s, 1H), 7.33-7.42 (m, 2H), 7.47-7.54 (m, 2H), 7.54-7.59 (m, 1H), 7.65 (t, J=6.9 Hz, 1H), 7.98 (d, J=7.7 Hz, 2H), 8.25 (d, J=7.6 Hz, 2H).

### Step 6: Synthesis of 1-((4R,5R,7S,8R)-7-fluoro-8-hydroxy-7-(hydroxymethyl)-6-oxa-1-thiaspiro[3.4]octan-5-yl)pyrimidine-2,4(1H,3H)-dione 22

Intermediate 21 (700 mg, 1.4 mmol) was solubilized in NH₃ (7M in MeOH) (200 mL) and stirred overnight at RT. The solvent was removed, and the solid was triturated in Et₂O to obtain compound 22 (269 mg, 65%).

MS (ES-): 303.0; ¹H NMR (400 MHz, DMSO-ds) δ ppm 2.32-2.45 (m, 1H), 2.83 (br dd, J=8.4, 4.0 Hz, 1H), 2.88-3.03 (m, 1H), 3.08-3.20 (m, 1H), 3.51-3.67 (m, 2H), 4.08 (br d, J=19.4 Hz, 1H), 5.67 (d, J=7.9 Hz, 1H), 5.75 (br s, 1H), 5.93 (br s, 1H), 6.71 (br s, 1H), 7.65 (br d, J=8.4 Hz, 1H), 11.53 (br s, 1H).

### Synthesis of (2S)-isopropyl 2-(((((4R,5R,7S,8R)-5-(2,4-dioxo-3,4-dihydropyrimidin-1(2H)-yl)-7-fluoro-8-hydroxy-6-oxa-1-thiaspiro[3.4]octan-7-yl)methoxy)(phenoxy)phosphoryl)-amino)propanoate 23

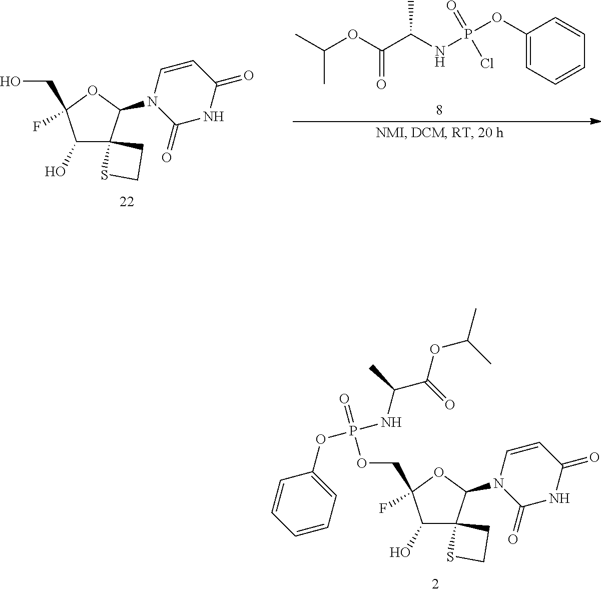 [SMILES: O=c1ccn([C@@H]2O[C@](F)(CO)[C@@H](O)[C@]23CCS3)c(=O)[nH]1 ; CC(C)OC(=O)[C@H](C)NP(=O)(Cl)Oc1ccccc1 ; CC(C)OC(=O)[C@H](C)NP(=O)(OC[C@@]1(F)O[C@@H](n2ccc(=O)[nH]c2=O)[C@@]2(CCS2)[C@@H]1O)Oc1ccccc1]

Compound 22 (100 mg, 0.329 mmol) was dissolved in dry pyridine (5 mL) and the solvent was removed under reduced pressure. The foam obtained was solubilized in dichloromethane (5 mL) and N-methyl imidazole (0.131 mL, 1.03 g/mL, 1.643 mmol). To this mixture intermediate 8 (0.5 mL, 1 M, 0.5 mmol) was added dropwise under N₂ atmosphere at RT. After 5 h of stirring, another equivalent of intermediate 8 was added. After stirring overnight, the reaction mixture was quenched with a mixture of 20 mL of cold water and 20 mL of dichloromethane. The resulting mixture was acidified with HCl 1M until pH=4 and extracted with dichloromethane (3×50 mL). The organic layer was dried over Na₂SO₄, filtrated and the solvent was removed under reduced pressure to afford 400 mg of foam containing the compound. A purification was performed by Prep HPLC using method B to yield 2 (44 mg, yield 23%).

MS (ES-): 572.1; ¹H NMR (400 MHz, DMSO-ds) δ ppm 1.15 (d, J=6.2 Hz, 6H), 1.21 (dd, J=10.6, 7.3 Hz, 3H), 2.53-2.64 (m, 1H), 2.82-2.97 (m, 2H), 3.05 (br s, 1H), 3.72-3.85 (m, 1H), 4.14-4.34 (m, 3H), 4.85 (dt, J=12.5, 6.3 Hz, 1H), 5.58 (d, J=8.1 Hz, 1H), 6.02-6.19 (m, 2H), 6.67 (br s, 1H), 7.14-7.25 (m, 3H), 7.37 (br t, J=7.9 Hz, 3H), 10.86-11.82 (m, 1H).

### Compound 3

### Synthesis of 1-((4R,5R,7R,8R)-8-hydroxy-7-(hydroxymethyl)-6-oxa-1-thiaspiro[3.4]octan-5-yl)pyrimidine-2,4(1H,3H)-dione 7

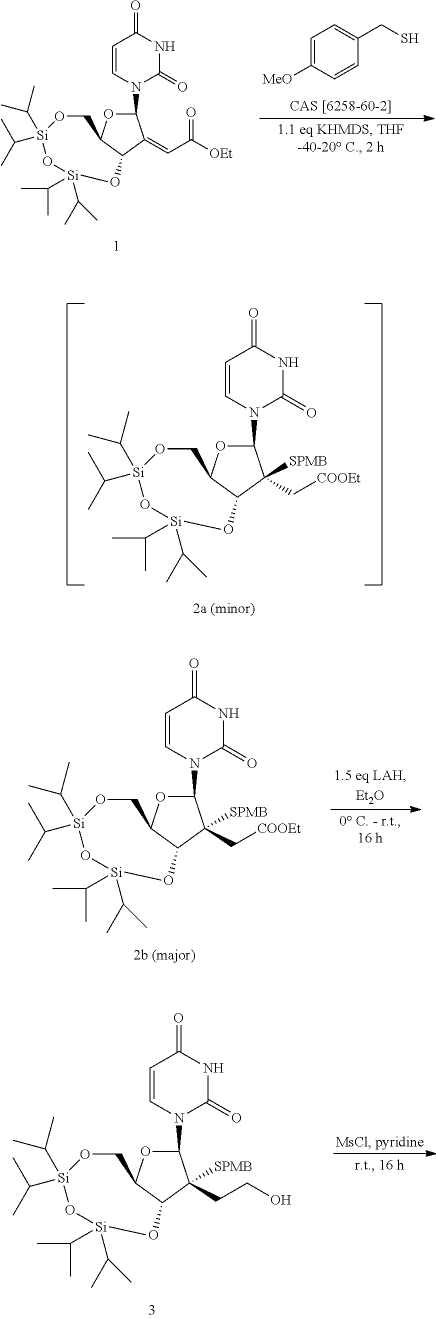

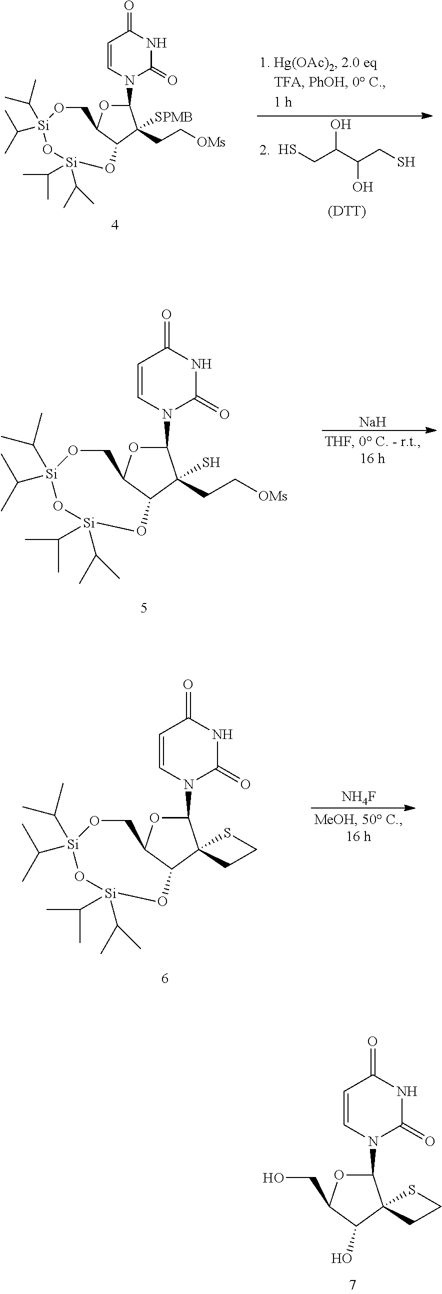

### Step 1: Synthesis of ethyl 2-((6aR,8R,9R,9aR)-8-(2,4-dioxo-3,4-dihydropyrimidin-1(2H)-yl)-2,2,4,4-tetraisopropyl-9-((4-methoxybenzyl)thio)tetrahydro-6H-furo[3,2-f][1,3,5,2,4]-trioxadisilocin-9-yl)acetate 2b

(4-Methoxyphenyl)methanethiol CAS[258-60-22](69.4 g, 450.6 mmol) in THF (5 L) was stirred at 20° C. under nitrogen. The mixture was cooled to −40° C. then KHMDS (1 M, 495.7 mL, 495.7 mmol) was added dropwise. The resulting white viscous liquid was stirred for 30 min then intermediate 1 (250 g, 450.6 mmol) in THF (1 L) was added at −40° C. The reaction mixture was allowed to warm slowly to 20° C. and stirred for 2 h. The reaction mixture was quenched by addition of aqueous solution 1N of HCl (2L) then extracted with EtOAc (2×2L). The organic layer was successively washed with aqueous solution of sodium bicarbonate (2 L), brine (2 L), dried over Na₂SO₄ and evaporated. The resulting residue was purified by column chromatography (PE/EA=20/1 to 3/1) to yield compound 2b (159 g, 50%) as colorless oil.

m/z=710 (M+H)⁺; ¹H NMR: (400 MHz, CDCl₃): δ8.26 (s, 1H), 7.82 (d, J=8.0 Hz, 1H), 7.29 (d, J=8.4 Hz, 2H), 6.85 (d, J=8.4 Hz, 2H), 6.28 (s, 1H), 5.66-5.63 (m, 1H), 5.34-5.30 (m, 1H), 4.41-4.23 (m, 1H), 4.19-4.04 (m, 5H), 3.80-3.78 (m, 4H), 3.23-3.19 (m, 1H), 2.92 (d, J=16.4 Hz, 1H), 1.30-0.86 (m, 51H).

### Step 2: Synthesis of 1-((6aR,8R,9R,9aR)-9-(2-hydroxyethyl)-2,2,4,4-tetraisopropyl-9-((4-methoxybenzyl)thio)tetrahydro-6H-furo[3,2-f][1,3,5,2,4]trioxadisilocin-8-yl)pyrimidine-2,4(1H,3H)-dione 3

Lithium aluminium hydride (4 g, 105 mml) was suspended in diethyl ether (1.5 L) under nitrogen at 0° C. then intermediate 2b (50 g, 70 mmol) in ether (200 mL) was added slowly at 0° C. The resulting white turbid solution was stirred at 20° C. for 16 h. The reaction mixture was quenched by addition of aqueous solution 1N of HCl (1 L) then extracted with EtOAc (2×1L). The organic layer was dried over Na₂SO₄ and evaporated. The resulting residue was purified by column chromatography (PE/EA=10/1 to 1/1) to yield compound 3 (27.8 g, 60%) as colorless oil.

m/z=668 (M+H)⁺; ¹H NMR: (400 MHz, CDCl₃): δ8.78 (s, 1H), 7.89 (d, J=8 Hz, 1H), 7.30 (d, J=8.4 Hz, 2H), 6.85 (d, J=8.8 Hz, 2H), 6.35 (s, 1H), 5.73 (d, J=8 Hz, 1H), 4.36-3.91 (m, 12H), 3.79 (s, 3H), 2.23-2.20 (m, 2H), 1.78-1.73 (m, 1H), 1.11-0.97 (m, 30H).

### Step 3: Synthesis of 2-((6aR,8R,9R,9aR)-8-(2,4-dioxo-3,4-dihydropyrimidin-1(2H)-yl)-2,2,4,4-tetraisopropyl-9-((4-methoxybenzyl)thio)tetrahydro-6H-furo[3,2-f][1,3,5,2,4]-trioxadisilocin-9-yl)ethyl methanesulfonate 4

Intermediate 3 (50 g, 75 mmol) was dissolved in pyridine (500 mL) under nitrogen at 25° C., then mesylchloride (12.8 g, 112.5 mmol) was slowly added at 25° C. The resulting yellow solution was stirred at 25° C. for 16 h. The reaction mixture was quenched by addition of aqueous solution 1N of HCl (1 L) then extracted with EtOAc (2×1 L). The organic layer was dried over Na₂SO₄ and evaporated. The resulting residue was purified by column chromatography (PE/EA=10/1 to 1/1) to yield compound 4 (43 g, 78%) as colorless oil.

m/z=746 (M+H)⁺

¹H NMR: (400 MHz, CDCl₃): δ8.56 (s, 1H), 7.92 (d, J=8.4 Hz, 1H), 7.31 (d, J=8.8 Hz, 2H), 6.87-6.85 (m, 2H), 6.27 (s, 1H), 5.77-5.74 (m, 1H), 4.55-4.53 (m, 2H), 4.38-4.02 (m, 8H), 3.79 (s, 3H), 2.95 (s, 3H), 2.28-2.21 (m, 1H), 1.12-1.01 (m, 31H).

### Step 4: Synthesis of 2-((6aR,8R,9R,9aR)-8-(2,4-dioxo-3,4-dihydropyrimidin-1(2H)-yl)-2,2,4,4-tetraisopropyl-9-mercaptotetrahydro-6H-furo[3,2-f][1,3,5,2,4]trioxadisilocin-9-yl)ethyl methanesulfonate 5

To intermediate 4 (62 g, 83.2 mmol) in TFA (250 mL) at 25° C., mercury acetate (53 g, 166.4 mmol) and phenol (39.1 g, 416 mmol) were added slowly at 0° C. The resulting dark red solution was stirred at 0° C. for 1 h. The 1,4-dimercaptobutane-2,3-diol (25.6 g, 166.4 mmol) was added at 0° C. The resulting mixture was stirred 10 min then filtered over Celite® and washed with ethylacetate (1 L). The pH was adjusted to 7 by addition of an aqueous solution of sodium bicarbonate. The resulting mixture was filtered over Celite® and extracted with EtOAc (2×1 L). The organic layer was dried over Na₂SO₄ and evaporated at 25° C. to give intermediate 5 (64 g, crude) as brown oil.

### Step 5: Synthesis of 1-((2′R,6aR,8R,9aR)-2,2,4,4-tetraisopropyltetrahydrospiro[furo[3,2-f]-[1,3,5,2,4]trioxadisilocine-9,2′-thietan]-8-yl)pyrimidine-2,4(1H,3H)-dione 6

Intermediate 5 (57 g, 91 mmol) was dissolved in THF (500 mL) at 20° C. under nitrogen. The resulting mixture was stirred at 0° C. then sodium hydride (3.6 g, 135 mmol) was added slowly. The reaction mixture was stirred at 20° C. for 16 h. The reaction mixture was quenched by addition of aqueous solution 1N of HCl (1 L) then extracted with EtOAc (2×1 L). The organic layer was dried over Na₂SO₄ and evaporated. The resulting residue was purified by column chromatography (PE/EA=10/1 to 5/1) to yield compound 6 (18.3 g, 46%, 2 steps) as colorless oil.

m/z=529 (M+H)⁺

¹H NMR: (400 MHz, CDCl₃): δ8.55 (s, 1H), 7.93 (d, J=8 Hz, 1H), 6.58 (s, 1H), 5.69 (d, J=8 Hz, 1H), 4.20-4.17 (m, 1H), 4.05-3.96 (m, 2H), 3.54-3.51 (m, 1H), 3.33-3.32 (m, 1H), 2.96-2.87 (m, 2H), 2.85-2.69 (m, 1H), 1.17-0.98 (m, 30H).

### Step 6: Synthesis of 1-((4R,5R,7R,8R)-8-hydroxy-7-(hydroxymethyl)-6-oxa-1-thiaspiro[3.4]-octan-5-yl)pyrimidine-2,4(1H,3H)-dione 7

Intermediate 6 (50 g, 94.5 mmol) was dissolved in methanol (500 mL) at 20° C. under nitrogen. Ammonium fluoride (10.5 g, 283.6 mmol) was added at 20° C. The reaction mixture was stirred at 50° C. for 16 h. The reaction mixture was allowed to cool down to RT then the solvent was removed under reduced pressure. The resulting residue was purified by column chromatography (DCM/MeOH: 100/1 to 10/1) to yield (11.2 g, 42%) of 7 as white solid.

m/z=287 (M+H)⁺

¹H NMR: (400 MHz, DMSO-ds): δ8.00 (d, J=8 Hz, 1H), 6.39 (s, 1H), 5.68 (d, J=6.4 Hz, 1H), 5.61 (d, J=8.4 Hz, 1H), 5.22 (t, J=4.8 Hz, 1H), 3.92-3.89 (m, 1H), 3.72-3.71 (m, 1H), 3.59-3.57 (m, 1H), 3.38 (d, J=8.4 Hz, 1H), 3.12-2.94 (m, 1H), 2.85-2.81 (m, 2H), 2.47-2.44 (m, 1H).

### Synthesis of (2S)-isopropyl 2-(((((4R,5R,7R,8R)-5-(2,4-dioxo-3,4-dihydropyrimidin-1(2H)-yl)-8-hydroxy-6-oxa-1-thiaspiro[3.4]octan-7-yl)methoxy)(phenoxy)phosphoryl)amino)propanoate

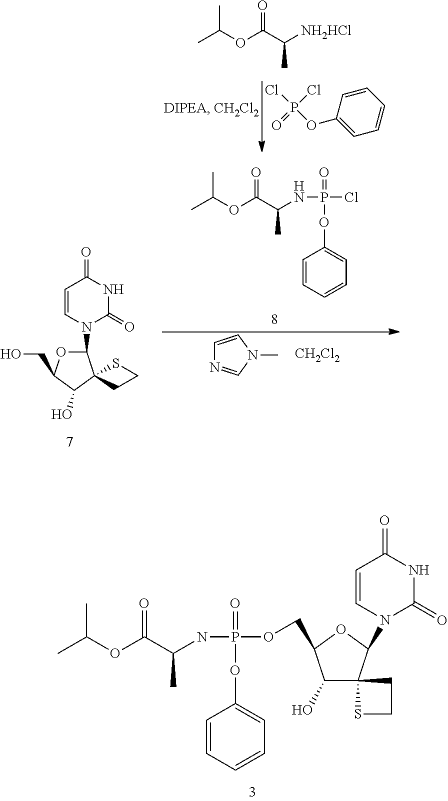

### Synthesis of 8

To a solution of (S)-isopropyl 2-aminopropanoate hydrochloride (5 g, 29.8 mmol) in dichloromethane (50 mL), phenyl phosphorodichloridate (4.45 g, 29.8 mmol) was added at 20° C. The resulting mixture was cooled to −78° C. then diisopropylethyl amine (10.4 mL, 59.6 mmol) was added dropwise. The reaction mixture was stirred at −78° C. for 1 h then the temperature of the reaction was allowed to rise to 20° C. After 1 h the solvent was removed under reduced pressure.

Dry Et₂O (about 50 ml) was added and the formed precipitate was filtered off and washed two times with dry Et₂O under nitrogen. The filtrate was evaporated to dryness to give yellow colorless oil 8 (8.32 g) which was stored as a 1 M solution in dry tetrahydrofuran (THF) in the freezer at −20° C.

¹H NMR (400 MHz, CDCl₃) δ ppm 1.24-1.31 (m, 6H), 1.50 (dd, J=7.0, 2.1 Hz, 3H), 4.06-4.20 (m, 1H), 4.23-4.41 (m, 1H), 5.02-5.14 (m, 1H), 7.19-7.30 (m, 3H), 7.34-7.41 (m, 2H).

### Synthesis of 3

Compound 7 (500 mg, 1.7 mmol) was dissolved in dry pyridine (15 mL) and stirred for 1 h at RT then evaporated to dryness.

The resulting precipitate was suspended in dry dichloromethane (15 mL) and methyl imidazole (1.3 mL, 17.4 mmol) was added dropwise. The resulting solution was treated with phosphorochloridate 8 (2.62 mL, 2.62 mmol) 1 M solution in dry THF under nitrogen. The reaction mixture was stirred at 20° C. for 16 h and was diluted with DCM (20 mL) and washed with aqueous solution of 1M HCl (3×20 mL). The combined aqueous layers were extracted with DCM (30 mL). The combined organic layers were dried over Na₂SO₄, filtered and concentrated. The residue was purified by column chromatography (gradient DCM/MeOH 1 to 10%) to yield (100 mg, 12%) of 3 as white foam.

m/z=556 (M+H)⁺; ¹H NMR (400 MHz, CDCl₃) δ ppm 1.18-1.26 (m, 6H), 1.30-1.38 (m, 2H), 2.62 (s, 1H), 2.66-2.90 (m, 2H), 3.01 (td, J=8.7, 5.6 Hz, 1H), 3.14-3.22 (m, 1H), 3.46 3.63 (m, 1H), 3.70 (s, 1H), 3.85-4.04 (m, 3H), 4.32-4.54 (m, 2H), 4.97-5.07 (m, 1H), 5.59-5.65 (m, 1H), 6.50-6.55 (m, 1H), 7.15-7.25 (m, 3H), 7.30-7.37 (m, 2H), 7.46-7.55 (m, 1H), 9.07 (br s, 1H).

Compound 4

HPLC Condition A, Rt: 1.88 min, m/z=554 (M+H)⁺; ¹H NMR (400 MHz, DMSO-d₆) □ ppm 0.77-0.93 (m, 3H), 1.14-1.25 (m, 3H), 1.24-1.37 (m, 2H), 1.41-1.62 (m, 2H), 2.35-2.47 (m, 2H), 3.63-3.92 (m, 3H), 3.92-4.06 (m, 2H), 4.05-4.21 (m, 1H), 4.23-4.46 (m, 3H), 5.48-5.59 (m, 1H), 5.59-5.72 (m, 1H), 5.89-6.15 (m, 2H), 7.09-7.25 (m, 3H), 7.31-7.41 (m, 2H), 7.43-7.52 (m, 1H), 11.51 (br. s., 1H)

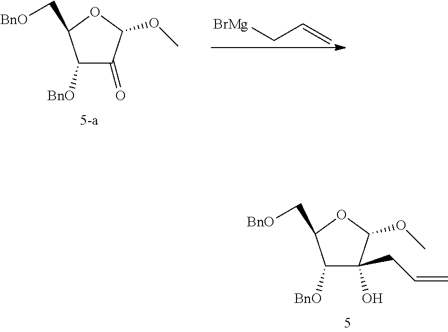

Under argon atmosphere, to a solution of 5a (obtained as in Org. Lett., 2007, 9, 3009-3012) in dry tetrahydrofurane (THF; 400 mL) at −78° C., allylmagnesium bromide (400 mL, 400 mmol; 1.0 M in diethylether) was added. After stirring the reaction mixture at −78° C. for 4 hours, the reaction mixture was allowed to stir at room temperature for 2 hours. The reaction was carefully quenched with saturated aqueous ammonium chloride. The mixture was extracted with dichloromethane, and the organic layer was washed with brine. The solvent was removed, and the residue was purified by silica gel chromatography (600 g silica), by gradient elution with 15% to 20% ethyl acetate in hexane to give the reaction product 5 as a colorless oil (32.9 g, 70%).

HPLC Condition A, Rt: 2.97 min, m/z=402 (M+NH₄)⁺. ¹H NMR (400 MHz, CDCl₃) δ ppm 7.38-7.20 (m, 1OH), 5.84-5.97 (m, 1H), 5.12 (d, 1H, J=10.2 Hz), 5.01 (d, 1H, J=17.2 Hz), 4.74 (d, 1H, J=12.3 Hz), 4.56 (s, 1H), 4.53-4.40 (m, 3H), 4.05-4.11 (m, 1H), 3.32-3.53 (m, 4H), 3.44 (s, 3H), 2.37 (dd, 1H, J=14.3, 6.7 Hz), 2.25 (dd, 1H, J=14.3, 7.6 Hz).

### (2S,3R,4R,5R)-3-allyl-4-(benzyloxy)-5-(benzyloxymethyl)-2-methoxytetrahydrofuran-3-yl benzoate (6)

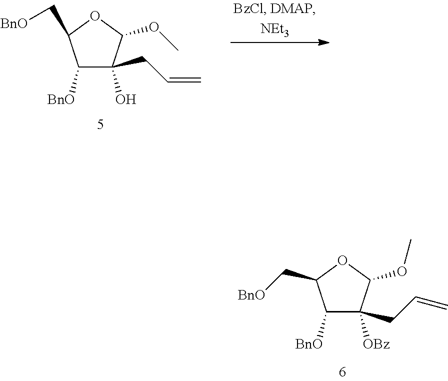

To a solution of 5 (26.6 g, 69.2 mmol) in dry dichloromethane (500 mL) at room temperature, N,N-dimethylpyridin-4-amine (DMAP; 2.113 g, 17.30 mmol), triethylamine (217 mL, 1557 mmol) and benzoyl chloride (18.05 mL, 156 mmol) were added. After 1 hour, additional benzoyl chloride (6 mL) and DMAP (2.1 g) were added. The mixture was stirred for 5 days. The reaction mixture was then stirred with 1N HCl and extracted with dichloromethane. The organic layers were combined and washed with saturated aqueous NaHCO₃ followed by brine. After drying with MgSO₄, filtration and evaporation of the volatiles, the residue was purified by column chromatography (400 g silica) eluting with heptane to 15% ethyl acetate in heptane to give reaction product as an oil (as a mixture with compound 5). The mixture was purified again with CH₂Cl₂ as eluent (400 g silica). The pure fractions were collected and intermediate 6 was obtained as a colorless oil (13.05 g, 39%). HPLC Condition A, Rt: 3.41 min, m/z=457 (M-OMe)⁺. ¹H NMR (400 MHz, CDCl₃) δ ppm 8.1 (d, 2H, J=7.9 Hz), 7.68-7.28 (m, 13H), 5.84-5.77 (m, 1H), 5.12 (d, 1H, J=16 Hz), 4.95 (d, 1H, J=16 Hz), 4.92 (d, 1H, J=12.3 Hz), 4.56 (d, 1H, J=12.3 Hz), 4.48 (d, 1H, J=11.6 Hz), 4.40 (d, 1H, J=11.6 Hz), 4.2 (m, 1H), 3.85 (d, 1H, J=6.2 Hz), 3.53 (d, 1H, J=10.8 Hz), 3.7 (s, 3H), 3.45 (dd, 1H, J=10.8, 6.2 Hz), 3.25 (dd, 1H, J=15.5, 7.3 Hz), 2.45 (dd, 1H, J=15.5, 7.3 Hz).

### 1-[(2R,3R,4R,5R)-3-allyl-4-(benzyloxy)-5-(benzyloxymethyl)-3-hydroxytetrahydrofuran-2-yl]pyrimidine-2,4(1H,3H)-dione (7)

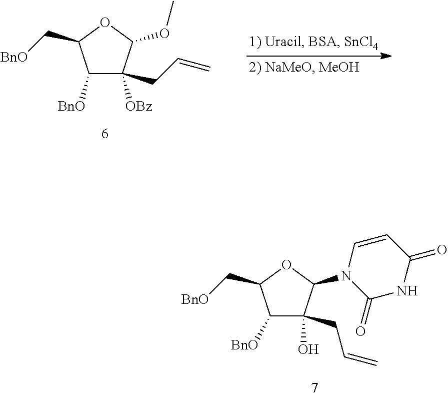

Bis(trimethylsilyl)acetamide (BSA; 29.2 mL, 118 mmol) was added to a mixture of 6 (14.0 g, 23.1 mmol) and uracil (5.99 g, 53.4 mmol) in anhydrous acetonitrile (300 mL). The reaction mixture was refluxed for 1 hour and the clear solution was allowed to cool down to room temperature. Tinchloride (11.55 mL, 99 mmol) was added dropwise at room temperature and the mixture was further stirred for 1 hour. The mixture was then stirred at reflux for 1.5 hour and again cooled to room temperature. Ethyl acetate (250 mL) was added, followed by saturated aqueous NaHCO₃ (250 mL) and the mixture was stirred for 15 minutes. After filtration through Celite, the organic layer was separated and washed with saturated aqueous NaHCO₃ (250 mL). The combined aqueous layer was extracted with ethyl acetate (250 mL) and the combined organic layer was dried (MgSO₄), filtered and evaporated to dryness under reduced pressure. The resulting yellow oil was dissolved in methanol and 25% sodium methanolate (25 mL) was added. Stirring continued overnight. More 25% sodium methanolate (15 mL) was added and stirring was continued overnight. Acetic acid (30 mL) was added and the solvent was removed. The residue was purified by column chromatography with heptane/ethyl acetate 50:50 to 100% ethyl acetate. Intermediate 7 (9.38 g, 76%) was obtained as a colorless oil. HPLC Condition A, Rt: 2.49 min, m/z=465 (M+H)⁺. ¹H NMR (400 MHz, CDCl₃) δ ppm 8.39 (1H, NH), 7.75 (d, 1H, J=8.0 Hz), 7.22-7.43 (m, 1OH), 6.05 (s, 1H), 5.71-5.84 (m, 1H), 5.35 (d, 1H, J=8.0 Hz), 5.00-5.11 (m, 2H), 4.70 (d, 1H, J=11.5 Hz), 4.53 (d, 1H, J=11.5 Hz), 4.47 (d, 1H, J=11.1 Hz), 4.47 (d, 1H, J=11.1 Hz), 4.11-4.16 (m, 1H), 4.04 (d, 1H, J=8.0 Hz), 3.81-3.87 (m, 1H), 3.45-3.52 (m, 1H), 3.17 (bs, OH), 2.15-2.33 (m, 2H).

### 1-[(2R,3R,4R,5R)-4-(benzyloxy)-5-(benzyloxymethyl)-3-hydroxy-3-(2-hydroxyethyl)tetrahydrofuran-2-yl]pyrimidine-2,4(1H,3H)-dione (8)

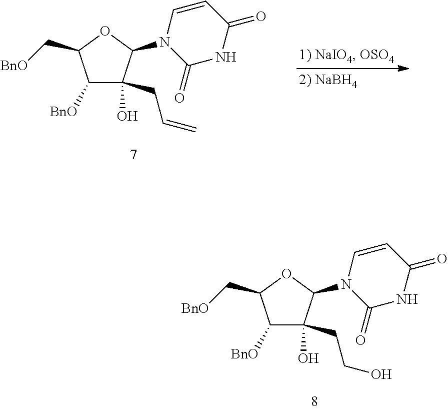 [SMILES: C=CC[C@@]1(O)[C@H](OCc2ccccc2)[C@@H](COCc2ccccc2)O[C@H]1n1ccc(=O)[nH]c1=O ; O=c1ccn([C@@H]2O[C@H](COCc3ccccc3)[C@@H](OCc3ccccc3)[C@]2(O)CCO)c(=O)[nH]1]

To a stirred solution of 7 (7.8 g, 16.79 mmol) in a mixture of THF (10 mL) and H₂O (10 mL) was added sodium periodate (11.17 g, 52.2 mmol) followed by osmium(VIII) tetroxide (2 mL, 2.5 w/v % in tert-Butanol, 0.168 mmol) and stirring was continued for 2 hour at room temperature. Water (100 mL) was added and extraction was performed with ethyl acetate (2×50 mL). The organic layer was washed with saturated aqueous NaHCO₃ (2×30 mL). The combined aqueous layer was extracted with ethyl acetate and the combined organic layer was dried over (Na₂SO₄), filtered and evaporated to dryness under reduced pressure. The oily residue obtained was dissolved in a mixture of THF (100 mL) and H₂O (20 mL) and sodium borohydride (1.361 g, 36.0 mmol) was added. The reaction mixture was stirred overnight at room temperature, whereupon water (100 mL) was added and extraction was performed with ethyl acetate (2×50 mL). The combined organic layer was washed with saturated aqueous NaHCO₃, the combined aqueous layer was extracted with ethyl acetate, and the combined organic layer was dried over (Na₂SO₄), filtered and evaporated to dryness under reduced pressure. The oily residue obtained was purified by column chromatography (0-10% (v/v) methanol in CH₂Cl₂ then 10% isocratic) affording reaction product 8 as a white foam (4.8 g, 57%). HPLC Condition A, Rt: 2.12 min, m/z=469 (M+H)⁺. ¹H NMR (400 MHz, CDCl₃) δ ppm 9.85 (1H, NH), 7.85 (d, 1H, J=8.0 Hz), 7.22-7.43 (m, 10H), 6.05 (s, 1H), 5.35 (d, 1H, J=8.0 Hz), 4.75 (d, 1H, J=11.5 Hz), 4.53 (d, 1H, J=11.5 Hz), 4.45 (d, 1H, J=11.3 Hz), 4.35 (d, 1H, J=11.3 Hz), 4.27 (d, 1H, J=6.6 Hz), 4.2 (s, 1H), 4.1, (d, 1H, J=6.6 Hz), 3.95 (d, 1H, J=10.8 Hz), 3.75-3.7 (m, 1H), 3.62 (d, 1H, J=10.8 Hz), 3.17 (bs, OH), 1.8-1.7 (m, 2H).

### 1-[(4R,5R,7R,8R)-8-(benzyloxy)-7-(benzyloxymethyl)-1,6-dioxaspiro[3.4]octan-5-yl]pyrimidine-2,4(1H,3H)-dione (9)

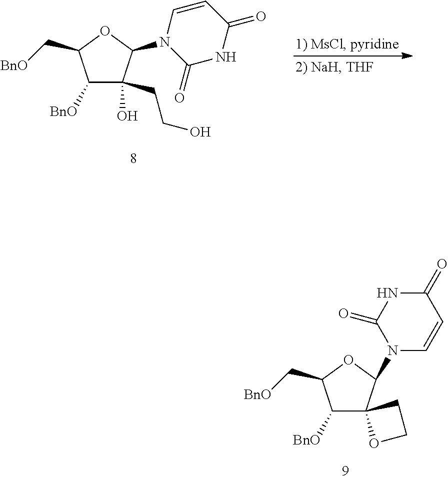 [SMILES: O=c1ccn([C@@H]2O[C@H](COCc3ccccc3)[C@@H](OCc3ccccc3)[C@]2(O)CCO)c(=O)[nH]1 ; O=c1ccn([C@@H]2O[C@H](COCc3ccccc3)[C@@H](OCc3ccccc3)[C@]23CCO3)c(=O)[nH]1]

Methanesulfonyl chloride (0.800 mL, 10.34 mmol) was added to 8 (4.32 g, 9.22 mmol) in dry pyridine (100 mL). After 1 hour and 15 minutes, 0.1 equivalents more methanesulfonyl chloride was added and the mixture was further stirred at room temperature for 45 minutes. Then, a small amount of methanol was added and the mixture was evaporated to dryness. The residue was dissolved in ethyl acetate (100 mL) and washed with saturated NaHCO₃ (2×50 mL). The combined aqueous layer was extracted with ethyl acetate. The combined organic layer was then dried over Na₂SO₄ and concentrated in vacuo. The obtained residue was dissolved in dry THF and 95% NaH (932 mg, 36.9 mmol) was added at once at room temperature. After stirring for 2 hours at room temperature, the reaction mixture was poured on a saturated aqueous solution of NH₄Cl (30 mL) followed by addition of CH₂Cl₂ (250 mL). The separated organic layer was washed with saturated aqueous NaHCO₃ (2×100 mL) and the combined aqueous layer was extracted with CH₂Cl₂ (250 mL). The combined organic layer was dried (Na₂SO₄), filtered and evaporated to dryness under reduced pressure. The residue obtained was purified by column chromatography eluting first with heptane, then with ethyl acetate to afford 9 (3.27 g, 79%) as a foam. HPLC Condition A, Rt: 2.33 min, m/z=451 (M+H)⁺. ¹H NMR (400 MHz, CDCl₃) δ ppm 2.20-2.38 (m, 1H) 2.38-2.52 (m, 1H) 3.62 3.73 (m, 1H) 3.89-4.13 (m, 3H) 4.38-4.56 (m, 3H) 4.56-4.68 (m, 1H) 4.70-4.88 (m, 2H) 5.25 (d, J=8.00 Hz, 1H) 6.25 (s, 1H) 7.18-7.47 (m, 10H) 7.87 (d, J=8.20 Hz, 1H) 8.90 (br. s., 1H)

### 1-[(4R,5R,7R,8R)-8-hydroxy-7-(hydroxymethyl)-1,6-dioxaspiro[3.4]octan-5-yl]pyrimidine-2,4(1H,3H)-dione (10)

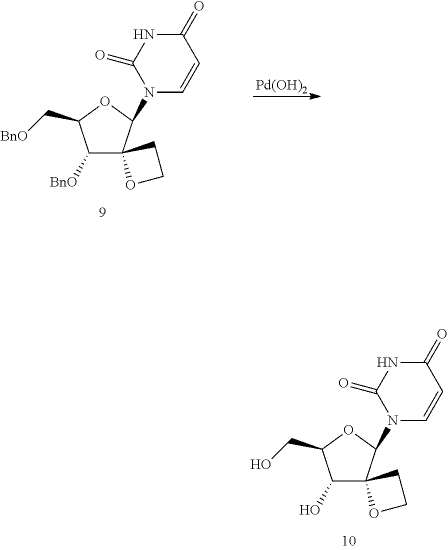

A mixture of 9 (50 mg, 0.111 mmol) in methanol (1 mL) and Pd(OH)₂ (8 mg) was stirred under a hydrogen atmosphere at room temperature. After 4 hours, more Pd(OH)₂ (30 mg) and methanol (1 mL) were added. The mixture was stirred vigorously under H₂-atmosphere overnight. The catalyst was removed by filtration over decalite, and the solvent was removed by evaporation. The resulting residue was purified by silica gel chromatography eluted with 10% methanol in ethyl acetate to give the intermediate 10 as white powder (16.8 mg; 56%). HPLC Condition B, Rt: 1.98 min, m/z=271 (M+H)⁺. ¹H NMR (400 MHz, D₂O) δ ppm 7.65 (d, 1H, J=8.0 Hz), 6.11 (s, 1H), 5.82 (d, 1H, J=8.0 Hz), 4.46-4.61 (m, 2H), 4.06-4.13 (m, 1H), 3.87-3.95 (m, 1H), 3.69-3.77 (m, 2H), 2.62-2.73 (m, 1H), 2.48-2.58 (m, 1H).

### methyl 2-(chloro(phenoxy)phosphorylamino)-2-methylpropionate (11)

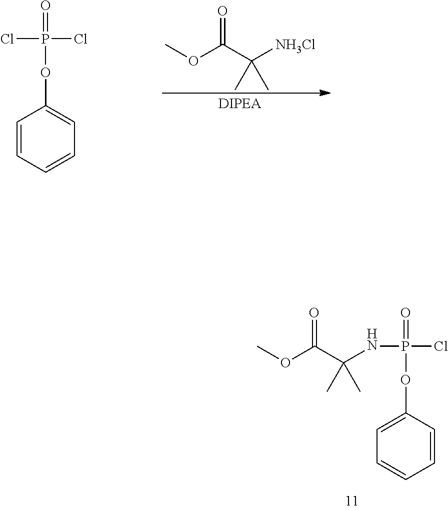

A solution of phenylphosphorodichloridate (1.0 eq., 13.0 mmol, 1.9 mL) and methyl α-aminoisobutyrate hydrochloride (1.0 eq., 13.0 mmol, 2.0 g) in CH₂Cl₂ (80 mL) was cooled to −80° C. Dry N,N-diisopropylethylamine (DIPEA; 2.0 eq., 26.0 mmol, 4.3 mL) was added dropwise. After 2 hours, the reaction was warmed to room temperature and the solvent was removed under reduced pressure. Dry diethylether was added and the precipitate was filtered off and washed twice with dry diethylether under an argon atmosphere. The filtrate was evaporated to dryness to give 11 which was stored as a 0.90 M solution in dry tetrahydrofuran (THF) at −18° C.

A solution of phenylphosphorodichloridate (1.0 eq., 13.0 mmol, 1.9 mL) and methyl α-aminoisobutyrate hydrochloride (1.0 eq., 13.0 mmol, 2.0 g) in CH₂Cl₂ (80 mL) was cooled to −80° C. Dry N,N-diisopropylethylamine (DIPEA; 2.0 eq., 26.0 mmol, 4.3 mL) was added dropwise. After 2 hours, the reaction was warmed to room temperature and the solvent was removed under reduced pressure. Dry diethylether was added and the precipitate was filtered off and washed twice with dry diethylether under an argon atmosphere. The filtrate was evaporated to dryness to give 11 which was stored as a 0.90 M solution in dry tetrahydrofuran (THF) at −18° C.

### methyl 2-[[[(4R,5R,7R,8R)-5-(2,4-dioxo-3,4-dihydropyrimidin-1(2H)-yl)-8-hydroxy-1,6-dioxaspiro[3.4]octan-7-yl]methoxy](phenoxy)phosphorylamino]-2-methylpropanoate (4)

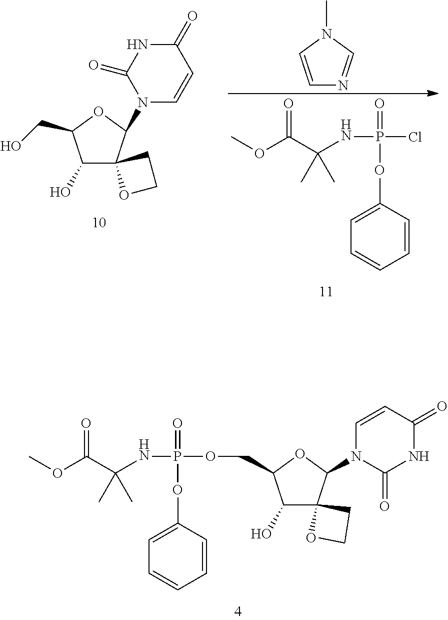

To a solution of 10 (1.0 eq., 0.28 mmol, 75 mg) in dry THF (3 mL) was added 1-methylimidazole (NMI; 12.0 eq., 3.33 mmol, 0.27 mL) at room temperature. A solution of intermediate 11 (1.4 eq., 0.39 mmol, 0.43 mL) was added dropwise and the mixture was stirred at room temperature for 1 hour. The reaction mixture was washed three times with 0.5 M HCl. The organic layer was dried over MgSO₄ and concentrated in vacuo. The residue was purified by column chromatography on silica gel (0-10% methanol in CH₂Cl₂) to give compound 4 (24 mg, yield=15%, purity=95%) as a mixture of diastereomers. HPLC Condition A; Rt: 1.49 min, m/z=526 (M+H)⁺. 1H NMR (400 MHz, DMSO-d₆) δ ppm 1.33 (s, 3H), 1.37 (s, 3H), 2.42-2.43 (m, 2H), 3.56 (s, 3H), 3.70-3.79 (m, 1H), 3.80-3.88 (m, 0.4H), 3.88-3.96 (m, 0.6H), 4.09-4.20 (m, 1H), 4.26-4.48 (m, 3H), 5.50-5.56 (m, 1H), 5.61-5.69 (m, 1H), 5.88-5.97 (m, 1H), 5.97-6.04 (m, 1H), 7.12-7.24 (m, 3H), 7.31-7.41 (m, 2H), 7.44 (d, J=8.22 Hz, 0.4H), 7.52 (d, J=8.02 Hz, 0.6H), 11.49 (br. s., 1H).

### Example 2: Biological Activity

The antiviral activity of compounds was tested against the rat HEV replicon LA-B350/luc as described in Debing et al (Dis Model Mech 2016; 9:1203-10). To this end, Huh7 cells were electroporated with capped viral RNA produced from plasmid pLA-B350/luc, plated in 96-well plates and treated with each of compounds at selected concentrations. For virus control (VC), compound was omitted. After 3 days, luminescence produced by the secreted Gaussia luciferase was quantified using the Promega Renilla luciferase kit and corrected for background with a cell control (CC, viral RNA and compound omitted). The 50% effective concentration (EC50) is defined as the concentration of compound that causes a 50% reduction in the Luc signal compared to that of average corrected VC. The EC50 was based on two or three experiments and derived by a nonlinear regression fit in GraphPad using a two-parameter logistic model while keeping the slope variable.

For viability evaluation, medium was removed and cells were subsequently incubated with MTS/PMS solution (3-(4,5-dimethylthiazol-2-yl)-5-(3-carboxymethoxyphenyl)-2-(4-sulfophenyl)-2H-tetrazolium/phenazinemethosulfate), which is metabolized to produce a brown, water-soluble product that is quantified after 1 h at 37° C. by absorbance read-out at 498 nm. Obtained values are expressed as percent inhibition of untreated RNA-transfected control condition. The CC50 represents the concentration at which the metabolic activity of the cells would be reduced to 50% of the metabolic activity of untreated cells (and was based on two experiments and derived by a nonlinear regression fit in GraphPad using a two-parameter logistic model while keeping the slope variable).

The results demonstrate that compounds of Formula (I) are active against HEV (rat HEV replicon LA-B350/luc).

| Compound number | EC₅₀ (μM) | CI95 % (μM) |
|---|---|---|
| 1 | 0.74 | 0.032 to 75 |
| 2 | 5.9 | 2.7 to 14 |
| 3 | 27 | 14 to 69 |
| 4 | 0.36 | 0.13 to 0.93 |

Reported values may be rounded to two significant figures

None of compounds 1-4 reached a CC50 at the highest tested concentration of 50 μM.

### Example 3: HEV Genotype 3 Replicon Kernow-C₁ p6/Luc

The antiviral activity of compounds was tested against the HEV Genotype 3 replicon Kernow-C₁ p6/luc (Kernow-C₁ p6: GenBank accession number JQ679013) as previously described (Debing Y, Emerson S U, Wang Y, Pan Q, Balzarini J, Dallmeier K, Neyts J. 2013. Ribavirin Inhibits In Vitro Hepatitis E Virus Replication through Depletion of Cellular GTP Pools and Is Moderately Synergistic with Alpha Interferon. Antimicrob Agents Chemother, 58:267-273). To this end, Huh7 cells were electroporated with capped in vitro transcribed Kernow-C₁ p6/luc-RNA produced from Mlul-digested plasmid DNA (Shukla P, Nguyen H T, Faulk K, Mather K, Torian U, Engle R E, Emerson SU. 2012. Adaptation of a genotype 3 hepatitis E virus to efficient growth in cell culture depends on an inserted human gene segment acquired by recombination. J. Virol. 86:5697-5707), seeded in 96-well plates containing serial dilutions of the test compounds. For virus control (VC) compound was omitted. After 4 days, luminescence produced by the secreted Gaussia luciferase was quantified using the Promega Renilla luciferase kit and corrected for background with a cell control (CC, viral RNA and compound omitted). The relative 50% effective concentration (EC50) is defined as the concentration of compound that causes a 50% reduction in the Luc signal, relative to the signal range. The relative EC50 was based on two experiments and derived by a nonlinear regression fit in GraphPad using a four-parameter logistic (4PL) model.

For viability evaluation, medium was removed and cells were subsequently incubated with MTS/PMS solution (3-(4,5-dimethylthiazol-2-yl)-5-(3-carboxymethoxyphenyl)-2-(4-sulfophenyl)-2H-tetrazolium/phenazinemethosulfate), which is metabolized to produce a brown, water-soluble product that is quantified after 1 h at 37° C. by absorbance read-out at 498 nm. Obtained values are expressed as percentage of untreated RNA-transfected control condition. The relative CC50 represents the concentration at which the metabolic activity of the cells would be reduced to 50% of the metabolic activity of untreated cells and was based on two or three experiments and derived by a nonlinear regression fit in GraphPad using a four-parameter logistic (4PL) model.

The results demonstrate that compounds of Formula (I) are active against HEV Genotype 3 replicon Kernow-C1 p6/luc.

| Compound number | relative EC₅₀ (μM) | CI95 % (μM) |
|---|---|---|
| 1 | >50 | NA |
| 3 | >50 | NA |
| 4 | 0.53 | 0,1529 to 25,86 |

Reported values may be rounded to two significant figures.

None of compounds 1-4 reached a relative CC50 at the highest tested concentration of 50 μM.

Compounds disclosed herein are potent and, without being bound by any theory, it is understood that this may translate in an in vivo setting to an effective therapy for the treatment of a hepatitis E infection.

### Example 4: In Vivo Efficacy in a HEV Athymic Nude Rats (HEV Strain LA-B350

Prior to the in vivo efficacy study, a new batch of the rat HEV virus is prepared from the livers of 10 infected athymic nude rats. This freshly prepared virus batch is used in all in vivo studies.

Thaw a vial containing 10% liver homogenate of the freshly prepared rat HEV. Dilute the virus stock ten times in PBS, corresponding to approximately 2×107 viral RNA copies. Infect the athymic nude rats with 200 μL of the diluted virus stock via intravenous injection in the tail vein. Start treating the rats 1 hour prior to infection and continue once daily with the treatment until day 14 pi. Weigh the rats and check for clinical signs every day until the end of the experiment (day 21 pi). From day 1-14 pi, collect blood once a week and faeces every 3 days to quantify the viral load by RT-qPCR. From day 15-21 pi, collect faeces every 3 days for quantification of the viral load. On day 21 pi, rats are euthanized via i.p injection of dolethal and blood collected via cardiac puncture and, upon intracardiac perfusion with PBS, the liver. Blood and liver are analysed for the presence of viral RNA (RT-qPCR) and histopathology.

| | Number | | | |
|---|---|---|---|---|
| Group | of rats | Dose (mg/kg) | Frequency | MOA |
| Vehicle | 6 | — | QD | Oral gavage |
| Ribavirin | 6 | 30 mg/kg | QD | i.p. |
| Test compound | 6 | High dose (e.g. 200 mg/kg) | QD | Oral gavage |
| Test compound | | Medium dose (e.g. 70 | QD | Oral gavage |
| | | mg/kg) | | |
| Test compound | 6 | Low dose (e.g. 20 mg/kg) | QD | Oral gavage |
| Sofosbuvir | 6 | To be determined | QD | Oral gavage |

| Day 0 | Day 1-14 pi | Day 15-20 pi | Day 21 pi |
|---|---|---|---|
| Weigh | Weigh | Weigh | Weigh |
| Collect | Check for clinical | Check for clinical | Check for clinical |
| faeces & | signs | signs | signs |
| blood | Treat once daily | Collect faeces every 3 | Sacrifice |
| Treat | Collect faeces every 3 | days | Collect blood, liver & |
| Infect | days | | faeces for RT-qPCR & |
| | Collect blood once a | | histology |
| | week | | |

Study design:
Day -1 or -2 pi: Divide 5-week-old (110-130 g) homozygous female athymic nude Hsd:RH-Foxn1rnu rats (Rattus norvegicus, Envigo, Horst, The Netherlands) into 4-6 groups (5 animals/group); give them an ear tag.
Day 0 pi: Weigh the rats and treat via oral gavage or ip (ribavirin or IFN), according to the schedule above, starting 1 h before infection. Intravenous infection with 200 μL of 1% liver homogenate of rat HEV strain LA-B350 (corresponding to approximately 2×107 viral RNA copies).
Day 1-14 pi: Weigh rats daily and treat once daily. Animals are monitored for mobility, care and behavior. Faeces will be collected every 3 days and blood (serum) will be collected once a week from the tail for quantification of viral load by RT-qPCR. When reaching humane endpoints (hunched back, ruffled fur, >20% weight loss, lethargic), animals will be euthanized.
Day 15-20 pi: Weigh rats daily. Animals are monitored for mobility, care and behavior. Faeces will be collected every 3 days for quantification of viral load by RT-qPCR. When reaching humane endpoints (hunched back, ruffled fur, ≥20% weight loss, lethargic), animals will be euthanized.
Day 21 pi: Animals are euthanized: collect liver, blood (serum) and faeces.

Sample processing:

Liver: 1) Quantification of viral load by RT-qPCR
2) Histological examination

Blood: Quantification of viral load by RT-qPCR

Faeces: Quantification of viral load by RT-qPCR

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A method of treating a hepatitis E virus (HEV) infection in a subject in need thereof, comprising administering a compound of formula (I) to the subject: 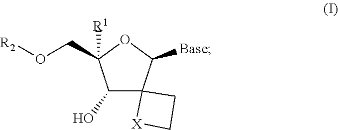
or a pharmaceutically acceptable salt thereof;
wherein:
Base is (b-1), (b-2), (b-3), (b-4), (b-5), (b-6), (b-7), or(b-8): 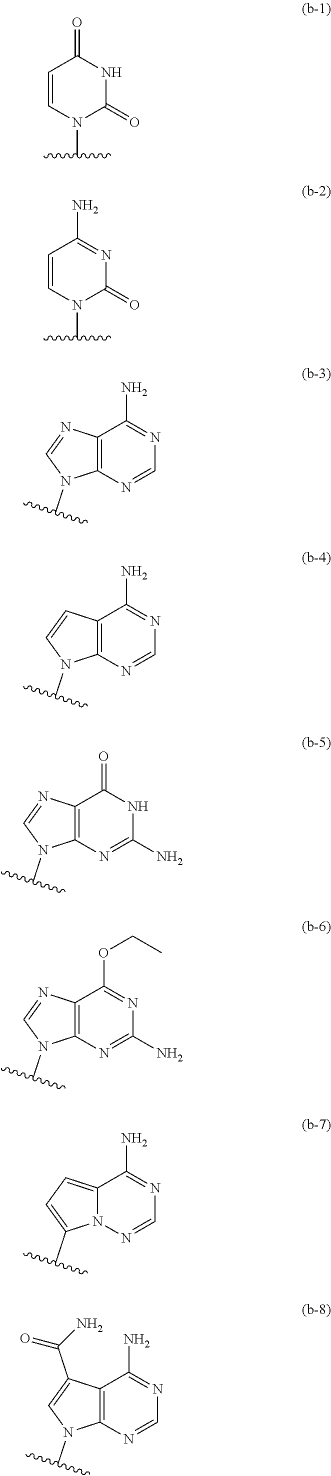
X is O or S;
R¹ is H, F, or N₃; and
R² is (f-1) or (f-2): 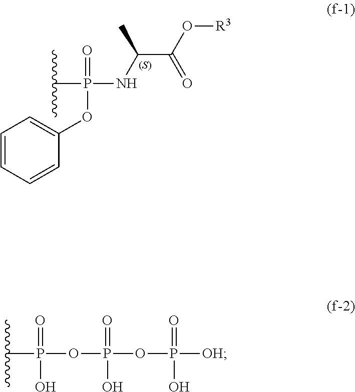
and
R³ is C₁₋₄alkyl.

2. The method of claim 1, wherein Base is (b-1).

3. The method of claim 1, wherein R² is (f-1).

4. The method of claim 1, wherein Base is (b-1), X is S, R² is (f-1) and R³ is isopropyl.

5. The method of claim 1, wherein Base is (b-1), X is O, R² is (f-1) and R³ is butyl.

6. The method of claim 1, wherein the compound is: 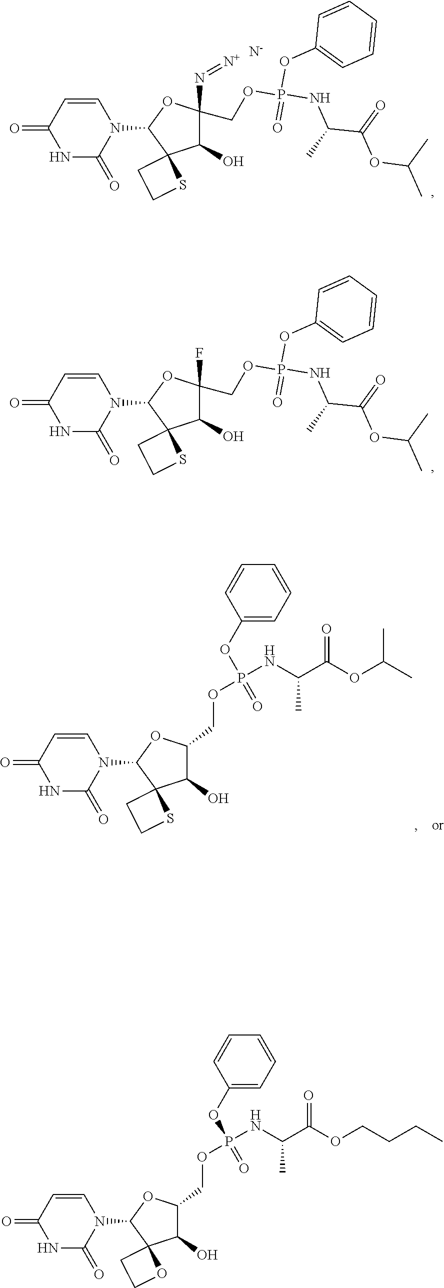
or a pharmaceutically acceptable salt thereof.

7. A pharmaceutical composition for treating a hepatitis E virus infection in a subject in need thereof, comprising administering a compound of formula (I) to the subject: 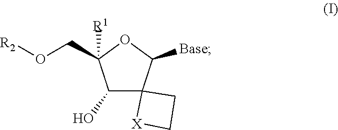
or a pharmaceutically acceptable salt thereof:
wherein:
Base is (b-1), (b-2), (b-3), (b-4), (b-5), (b-6), (b-7), or (b-8): 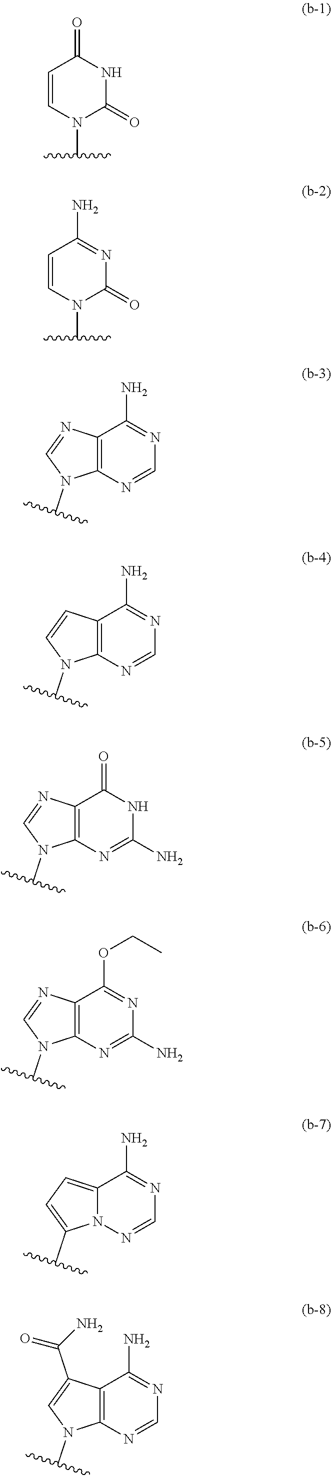
X is O or S;
R¹ is H, F, or N₃; and
R² is (f-1) or (f-2): 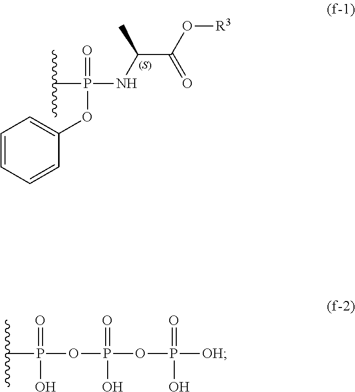
R³ is C₁-4alkyl;
and a pharmaceutically acceptable vehicle.

8. The method of claim 1, wherein the hepatitis E virus infection is a chronic HEV infection.

9. The method of claim 1, wherein the HEV virus infection is of genotype 1, genotype 2 or genotype 3.

10. The method of claim 1, wherein the subject is a pregnant woman, an immune-compromised subject or immune-deficient subject.

11. The pharmaceutical composition claim 7, wherein the hepatitis E virus infection is a chronic HEV infection.

12. The pharmaceutical composition of claim 7, wherein the HEV virus infection is of genotype 1, genotype 2 or genotype 3.

13. The pharmaceutical composition of claim 7, wherein the subject is a pregnant woman, an immune-compromised subject or immune-deficient subject.
